(19) United States
(12) Patent Application Publication
(10) Pub. No.: US 2024/0377418 A1
(43) Pub. Date: Nov. 14, 2024

(54) METHODS, DEVICES, AND RELATED ASPECTS FOR DETECTING CANNABINOIDS

(71) Applicant: ARIZONA BOARD OF REGENTS ON BEHALF OF ARIZONA STATE UNIVERSITY, Scottsdale, AZ (US)

(72) Inventors: Chao WANG, Chandler, AZ (US); MD Ashif IKBAL, Tempe, AZ (US)

(73) Assignee: ARIZONA BOARD OF REGENTS ON BEHALF OF ARIZONA STATE UNIVERSITY, Scottsdale, AZ (US)

(21) Appl. No.: 18/659,490

(22) Filed: May 9, 2024

Related U.S. Application Data
(60) Provisional application No. 63/501,773, filed on May 12, 2023.

(51) Int. Cl. G01N 33/94; B01L 3/00; G01N 33/543

(52) U.S. Cl. CPC G01N 33/948; B01L 3/50825; G01N 33/54346; G01N 33/54386; B01L 2300/0663; G01N 2333/415

(57) ABSTRACT

Provided herein are methods of detecting cannabinoid molecules in a sample. The methods include contacting the sample with a plurality of plasmonic metal nanoparticles (MNPs) that are conjugated with at least two sets of antibodies, or antigen binding portions thereof, that binds to at least first and second epitopes of the cannabinoid molecule under conditions sufficient for the antibodies, or the antigen binding portions thereof, to bind to the first and second epitopes of the cannabinoid molecule in the sample to produce bound cannabinoid molecules. The methods also include detecting the cannabinoid molecules when aggregations of MNPs coated with the target cannabinoid molecules form by connecting with one another. Related compositions, reaction mixtures, devices, kits, and systems are also provided.

### CROSS-REFERENCE TO RELATED APPLICATIONS

This application claims the benefit of U.S. Provisional Application No. 63/501,773 filed May 12, 2023, the disclosure of which is incorporated herein in its entirety.

### STATEMENT REGARDING FEDERALLY SPONSORED RESEARCH OR DEVELOPMENT

This invention was made with government support under 1847324 and 2020464 awarded by the National Science Foundation and R21 AI169098 awarded by the National Institutes of Health. The government has certain rights in the invention.

### BACKGROUND

The current gold standard detection method of small molecules, such as cannabinoids, like cannabidiol (CBD) and tetrahydrocannabinol (THC) molecules, is mass spectrometry. However, mass spectrometry is a costly and time-consuming process that demands professional personnel training, high-quality sample purification, and centralized facilities for data analysis, therefore unfeasible for many small-scale laboratories and clinics. In comparison, antibody-based detecting platforms suffer from the unavailability of binding epitopes to small molecules, and accordingly usually have relatively poor sensitivity and specificity. Recently, chemically induced dimerization (CID) methods have been developed to produce effective dimerization binders. However, small molecule detection, for example, on an enzyme-linked immunosorbent assay (ELISA) platform, is usually limited in sensitivity and speed due to slow mass transportation or surface reaction rate. In addition, series of sample purification, washing and amplification steps can seriously increase the detection time and cost.

Accordingly, there is a need for additional methods, and related aspects, of detecting small molecules, such as cannabinoids that are low cost, sensitive, easy-to-use, and which yield rapid point-of-care (POC) results, particularly under low resource conditions.

### SUMMARY

The present disclosure relates, in certain aspects, to ultra-sensitive gold nanoparticle (AuNP) and/or other plasmonic metal nanoparticle (MNP)-based colorimetric assays for cannabinoid detection. In some embodiments, the MNP surfaces are functionalized with high affinity antibodies, nanobodies, and/or other antigen binding portions to cannabinoid epitopes. In these embodiments, the cannabinoid epitopes generally induce the crosslinking of MNPs leading to large MNP aggregate formation and precipitation, resulting in a decrease of MNP monomer concentration in solution and accordingly, a visible change in solution transparency.

In one aspect, the present disclosure provides a method of detecting cannabinoid molecules (e.g., cannabidiol (CBD) molecules, tetrahydrocannabinol (THC) molecules, etc.) in a sample. In some embodiments, the method includes contacting the sample with a plurality of plasmonic metal nanoparticles (MNPs) (e.g., gold nanoparticles (AuNPs) or the like) that are conjugated (e.g., via a linker in some embodiments) with at least two sets of antibodies (e.g., monoclonal antibodies or the like), or antigen binding portions thereof, in which at least a first set of antibodies, or antigen binding portions thereof (e.g., an anchor binder), binds to a first epitope of a cannabinoid molecule and in which at least a second set of antibodies, or antigen binding portions thereof (e.g., a dimerization binder), binds to a second epitope of the cannabinoid molecule (e.g., the same cannabinoid molecule comprising the first epitope or a different cannabinoid molecule) under conditions sufficient for the first and second set of antibodies, or the antigen binding portions thereof, to bind to the first and second epitopes of the cannabinoid molecules in the sample to produce bound cannabinoid molecules. In some embodiments, the method includes contacting the sample with the plurality of MNPs conjugated with the first set of antibodies, or antigen binding portions thereof, (sometimes referred to herein as an “anchor binder”) under conditions sufficient for the first set of antibodies, or the antigen binding portions thereof, to bind to the first epitopes of the cannabinoid molecules in the sample to produce a first solution, and combining at least an aliquot of the first solution with the plurality of MNPs conjugated with the second set of antibodies, or antigen binding portions thereof, (sometimes referred to herein as a “dimerization binder”) under conditions sufficient for the second set of antibodies, or the antigen binding portions thereof, to bind to the second epitopes of the cannabinoid molecules in the aliquot of the first solution. In some embodiments, anchor binders have a higher binding affinity for the cannabinoid molecules than the dimerization binders. In some embodiments, the method includes contacting the sample with a plurality of plasmonic metal nanoparticles (MNPs) that are conjugated with a single set of antibodies, or antigen binding portions thereof, that bind to an identical epitope from different monomers of a dimerized cannabinoid molecule under conditions sufficient for the antibodies, or the antigen binding portions thereof, to bind to the identical epitope from the different monomers of the dimerized cannabinoid molecule in the sample to produce bound cannabinoid molecule. In some embodiments, the other MNPs comprise silver, copper, aluminum, platinum, palladium, or the like. In some embodiments, the sets of antibodies, or antigen binding portions thereof, comprise nanobodies. In some embodiments, a given antibody, or antigen binding portions thereof, comprises a mass of between about 10 kDa and about 20 kDa (e.g., about 11 kDa, about 12 kDa, about 13 kDa, about 14 kDa, about 15 kDa, about 16 kDa, about 17 kDa, about 18 kDa, or about 19 kDa). In some embodiments, a given antibody, or antigen binding portions thereof, comprises an equilibrium dissociation constant (K_{D}) on the order of between about 1 nM and about 100 nM (e.g., about 5 nM, about 10 nM, about 15 nM, about 20 nM, about 30 nM, about 40 nM, about 50 nM, about 60 nM, about 70 nM, about 80 nM, or about 90 nM). The method also includes detecting the cannabinoid molecules when one or more aggregations of the bound cannabinoid molecules form with one another, thereby detecting the cannabinoid molecules in the sample.

In some embodiments, the detection step comprises determining a change in absorbance at a resonance wavelength of the MNPs. In some embodiments, the method includes quantifying an amount of the cannabinoid molecules in the sample. In some embodiments, the method further includes centrifuging a solution comprising the sample and the plurality of MNPs to form MNP aggregations of the bound cannabinoid molecules prior to and/or during the detecting step. In some embodiments, the method further includes freezing the aggregations of the bound cannabinoid molecules prior to the detecting step. In some embodiments, the method includes drop casting the aggregations of the bound cannabinoid molecules prior to the detecting step. In some embodiments, the method includes obtaining the sample from a subject. In some embodiments, the method includes detecting the cannabinoid molecules within about 20 minutes or less of obtaining the sample from the subject. In some embodiments, the method includes repeating the method using one or more longitudinal samples obtained from the subject (e.g., to monitor the presence of the cannabinoid molecules in the subject over time, for example, as part of a drug screening protocol).

Essentially any sample type is used in performing the methods disclosed herein. In some embodiments, for example, the sample comprises blood, plasma, or serum. In some embodiments, the sample comprises saliva or sputum. In some embodiments, the sample comprises urine.

In some embodiments, the detecting step comprises measuring a colorimetric change when MNP aggregations of the bound cannabinoid molecules form with one another. In some embodiments, the method includes visually detecting the colorimetric change when the MNP aggregations of the bound cannabinoid molecules form with one another. In some embodiments, the method includes detecting the colorimetric change when the MNP aggregations of the bound cannabinoid molecules form with one another using a spectrometer. In some embodiments, a concentration of cannabinoid molecules in the sample is about 15 nM or less. In some embodiments, a concentration of cannabinoid molecules in the sample is about 100 pM or less. In some embodiments, the MNPs comprise a substantially spherical shape. In some embodiments, the MNPs comprise a cross-sectional dimension of between about 10 nm and about 100 nm.

In another aspect, the present disclosure provides a reaction mixture that includes a sample comprising cannabinoid molecules, and a plurality of plasmonic metal nanoparticles (MNPs) that are (i) conjugated with at least two sets of antibodies, or antigen binding portions thereof, wherein at least a first set of antibodies, or antigen binding portions thereof, binds to a first epitope of a cannabinoid molecule and wherein at least a second set of antibodies, or antigen binding portions thereof, binds to a second epitope of the cannabinoid molecule in the sample; or (ii) conjugated with a single set of antibodies, or antigen binding portions thereof, that bind to an identical epitope from different monomers of a dimerized cannabinoid molecule in the sample.

In another aspect, the present disclosure provides a composition that comprises a plurality of plasmonic metal nanoparticles (MNPs) that are (i) conjugated with at least two sets of antibodies, or antigen binding portions thereof, wherein at least a first set of antibodies, or antigen binding portions thereof, binds to a first epitope of a cannabinoid molecule and wherein at least a second set of antibodies, or antigen binding portions thereof, binds to a second epitope of the cannabinoid molecule in the sample; or (ii) conjugated with a single set of antibodies, or antigen binding portions thereof, that bind to an identical epitope from different monomers of a dimerized cannabinoid molecule in the sample.

In another aspect, the present disclosure provides a device that includes at least one reaction chamber (e.g., a body structure comprising an array of wells or the like) or substrate comprising a plurality of plasmonic metal nanoparticles (MNPs) that are (i) conjugated with at least two sets of antibodies, or antigen binding portions thereof, wherein at least a first set of antibodies, or antigen binding portions thereof, binds to a first epitope of a cannabinoid molecule and wherein at least a second set of antibodies, or antigen binding portions thereof, binds to a second epitope of the cannabinoid molecule when the reaction chamber or substrate receives a sample that comprises the cannabinoid molecule under conditions sufficient for the first and second set of antibodies, or the antigen binding portions thereof, to bind to the first and second epitopes of the cannabinoid molecules in the sample to produce bound cannabinoid molecules and one or more aggregations of the bound cannabinoid molecules to produce a colorimetric change in the reaction chamber; or (ii) conjugated with a single set of antibodies, or antigen binding portions thereof, that bind to an identical epitope from different monomers of a dimerized cannabinoid molecule under conditions sufficient for the antibodies, or the antigen binding portions thereof, to bind to the identical epitope from the different monomers of the dimerized cannabinoid molecule in the sample to produce bound cannabinoid molecules and/or one or more aggregations of the bound cannabinoid molecules to produce a colorimetric change in the reaction chamber. In some embodiments, the one or more aggregations of the bound cannabinoid molecules are drop cast in or on the reaction chamber or substrate. In some embodiments, a kit includes the device.

In another aspect, the present disclosure provides a system that includes a device, comprising at least one reaction chamber or substrate comprising a plurality of plasmonic metal nanoparticles (MNPs) (e.g., gold nanoparticles (AuNPs) or the like) that are (i) conjugated with at least two sets of antibodies, or antigen binding portions thereof, wherein at least a first set of antibodies, or antigen binding portions thereof, binds to a first epitope of a cannabinoid molecule and wherein at least a second set of antibodies, or antigen binding portions thereof, binds to a second epitope of a cannabinoid molecule when the reaction chamber receives a sample that comprises the cannabinoid molecule under conditions sufficient for the first and second set of antibodies, or the antigen binding portions thereof, to bind to the first and second epitopes of the cannabinoid molecules in the sample to produce bound cannabinoid molecules; or (ii) conjugated with a single set of antibodies, or antigen binding portions thereof, that bind to an identical epitope from different monomers of a dimerized cannabinoid molecule when the reaction chamber receives a sample that comprises the cannabinoid molecule under conditions sufficient for the antibodies, or the antigen binding portions thereof, to bind to the identical epitope from the different monomers of the dimerized cannabinoid molecule in the sample to produce bound cannabinoid molecules. The system also includes an electromagnetic radiation detection apparatus positioned, or positionable, within sufficient proximity to the device to detect one or more colorimetric changes produced in or on the reaction chamber or substrate when one or more aggregations of the bound cannabinoid molecules form with one another in or on the reaction chamber or substrate. In some embodiments, the electromagnetic radiation detection apparatus comprises a spectrometer. In some embodiments, the electromagnetic radiation detection apparatus comprises a microscope. In some embodiments, the electromagnetic radiation detection apparatus comprises a light-emitting diode (LED) that transmits light into and/or through the reaction chamber or substrate and a photodetector that detects light from the reaction chamber or substrate. In some embodiments, the system includes a device holder that is structured to hold the device, which device holder comprises at least one optical channel through which light is transmitted.

### BRIEF DESCRIPTION OF THE DRAWINGS

The accompanying drawings, which are incorporated in and constitute a part of this specification, illustrate certain embodiments, and together with the written description, serve to explain certain principles of the methods, reaction mixtures, devices, kits, and related systems disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1 is a flow chart that schematically shows exemplary method steps of detecting cannabinoid molecules in a sample according to some aspects disclosed herein.

FIG. 2 is a flow chart that schematically shows exemplary method steps of detecting cannabinoid molecules in a sample according to some aspects disclosed herein.

FIG. 3 is a schematic diagram of a work flow of an incubation-based CBD detection assay according to one exemplary embodiment.

FIG. 4 is a schematic diagram of a work flow of a centrifugation enhanced CBD detection assay according to one exemplary embodiment.

FIG. 5 is a schematic diagram of a work flow of a pre-incubated centrifuge enhanced CBD detection assay according to one exemplary embodiment.

FIGS. 6A and 6B schematically show aspects of an electronic detection system that comprises an LED circuit, a photodiode circuit, and a 3D printed Eppendorf tube holder.

FIGS. 7A-7C schematically show an overview of a small molecule sensor development method and readout methods. (a) Schematic of nano-sensor preparation. (b) Schematic of pre-incubated centrifuge enhanced CBD detection assay. (c) Schematics and images showing different readout methods for colorimetric and quantitative determination of antigen concentration.

FIGS. 8A-8H show a pre-incubated centrifuge enhanced detection of CBD molecule. (a) Final tube image of CBD detection in PBS with 4-hour incubation method. (b) Final tube image of CBD detection in PBS with centrifugation without pre-incubation. (c) Particle density/distribution across liquid height comparing different AuNP sizes with CBD molecule after centrifugation. (d) Schematic showing process flow of pre-incubated centrifuge enhanced detection of CBD molecule concertation sample. (e) Final tube image of CBD detection in PBS with centrifugation with pre-incubation (f) PDMS plate image of extracted top liquid from FIG. 8E. (g) Extinction spectra of AuNPs shown in FIG. 8F. (h) Extinction peak values (559 nm) extracted from FIG. 8G in black triangle and solid line compared to extinction peak values extracted from the top liquid of 4 hours incubated sample (FIG. 8A).

FIGS. 9A-9G show aspects of a size, structural and particle tracking analysis of detection assay. (a-d) Final tube images of CBD detection in PBS for AuNP sizes 40 nm (a), 60 (b), 80 (c) and 100 nm (d). (e) Extracted extinction peak for CBD detection in PBS for different nanoparticle sizes (f) AuNP size and population data for CBD detection in PBS for 80 nm AuNP extracted from NTA analysis. (g) TEM image showing single AuNPs from NC sample and AuNP cluster form 10 μM CBD concertation sample.

FIGS. 10A-10E show aspects of a modular analytic model for centrifugation enhanced nanoparticle-based antigen detection assay. (a) Schematic showing the functional difference between AuNP utilized centrifuge enhanced assay and typical affinity-based assay (b) Simulation response curve for showing the centrifugation enhancement effect. (c) Simulation response curve for showing the size effect. (d) Simulation response curve showing the effect of different binding affinities arising from different dimerization binding system. (e) Fitting of experimental data (black square dots) with simulation response curve (dashed line).

FIGS. 11A-11E show aspects of a simplified CBD assay design. (a) Schematic and extracted extinction peak for CBD detection in PBS for dimerization system CA14-CBD-DB21 (black triangle with solid line) and CA14-CBD-DB21 (square with dashed line). (b) Extracted extinction peak for CBD detection in PBS for filtered (black triangle with solid line) and unfiltered (square with dashed line) assay. (c) Schematic showing reaction constituents for filtered and unfiltered assay.

FIGS. 12A-12F show aspects of a rapid and electronic detection of CBD molecule in urine and saliva sample. (a) Schematic and visual image of electronic readout system, consisting mainly of a LED circuit, a photodiode circuit, and a 3D printed Eppendorf tube holder. (b) Final tube image of CBD detection in urine. (c) PDMS plate image of extracted top liquid from FIG. 12A. (d) Extinction spectra of AuNPs shown in FIG. 12B. (e) Extinction peak values (559 nm) extracted from FIG. 12D and plotted against CBD concentration shown as black square for CBD test in urine with solid line, solid circle with solid line for saliva along with THC detection with dashed lines black hollow square for urine and hollow circle for saliva. (f) Voltage signals measured in detecting CBD in 5% urine and saliva sample, shown as black circle for urine and hollow circle for saliva sample.

### DEFINITIONS

In order for the present disclosure to be more readily understood, certain terms are first defined below. Additional definitions for the following terms and other terms may be set forth throughout the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, reaction mixtures, devices, kits, and systems, the following terminology, and grammatical variants thereof, will be used in accordance with the definitions set forth below.

About. As used herein, “about” or “approximately” or “substantially” as applied to one or more values or elements of interest, refers to a value or element that is similar to a stated reference value or element. In certain embodiments, the term “about” or “approximately” or “substantially” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Antibody: As used herein, the term “antibody” refers to an immunoglobulin or an antigen-binding domain thereof. The term includes but is not limited to polyclonal, monoclonal, monospecific, polyspecific, non-specific, humanized, human, canonized, canine, felinized, feline, single-chain, chimeric, synthetic, recombinant, hybrid, mutated, grafted, and in vitro generated antibodies. The antibody can include a constant region, or a portion thereof, such as the kappa, lambda, alpha, gamma, delta, epsilon and mu constant region genes. For example, heavy chain constant regions of the various isotypes can be used, including: IgG₁, IgG₂, IgG₃, IgG₄, IgM, IgA₁, IgA₂, IgD, and IgE. By way of example, the light chain constant region can be kappa or lambda. The term “monoclonal antibody” refers to an antibody that displays a single binding specificity and affinity for a particular target, e.g., epitope.

Antigen Binding Portion: As used herein, the term “antigen binding portion” refers to a portion of an antibody that specifically binds to a cannabinoid molecule, such as a cannabidiol (CBD) molecule, tetrahydrocannabinol (THC) molecule, etc., e.g., a molecule in which one or more immunoglobulin chains is not full length, but which specifically binds to a targeted cannabinoid molecule. Examples of binding portions encompassed within the term “antigen-binding portion of an antibody include (i) a Fab fragment, a monovalent fragment consisting of the VLC, VHC, CL and CH1 domains: (ii) a F(ab′)2 fragment, a bivalent fragment comprising two Fab fragments linked by a disulfide bridge at the hinge region; (iii) a Fd fragment consisting of the VHC and CH1 domains; (iv) a Fv fragment consisting of the VLC and VHC domains of a single arm of an antibody, (v) a dAb fragment, which consists of a VHC domain; and (vi) an isolated complementarity determining region (CDR) having sufficient framework to specifically bind, e.g., an antigen binding portion of a variable region. An antigen binding portion of a light chain variable region and an antigen binding portion of a heavy chain variable region, e.g., the two domains of the Fv fragment, VLC and VHC, can be joined, using recombinant methods, by a synthetic linker that enables them to be made as a single protein chain in which the VLC and VHC regions pair to form monovalent molecules (known as single chain Fv (scFV)). Such single chain antibodies are also encompassed within the term “antigen binding portion” of an antibody. The term “antigen binding portion” encompasses a single-domain antibody (sdAb), also known as a “nanobody” or “VHH antibody,” which is an antibody fragment consisting of a single monomeric variable antibody domain. These antibody portions are obtained using conventional techniques known to those with skill in the art, and the portions are screened for utility in the same manner as are intact antibodies.

Bind: As used herein, “bind,” in the context of small molecule detection, refers to an attractive interaction between or among two or more molecules that produces a stable association in which the molecules are in close proximity to each other.

Cannabinoid: As used herein, “cannabinoid” or “cannabinoid molecule” refers to a ligand that is plant (e.g., cannabis plants) derived, synthetic, or semisynthetic, and has affinity for and activity at cannabinoid receptors. Examples of cannabinoids, include cannabichromenes (e.g., cannabichromene (CBC), cannabichromenic acid (CBCA), cannabichromevarin (CBCV), and cannabichromevarinic acid (CBCVA)), cannabicyclols (e.g., cannabicyclol (CBL), cannabicyclolic acid (CBLA), and cannabicyclovarin (CBLV)), cannabidiols (e.g., cannabidiol (CBD), cannabidiol monomethylether (CBDM), cannabidiolic acid (CBDA), cannabidiorcol (CBD-C1), cannabidivarin (CBDV), and cannabidivarinic acid (CBDVA)), cannabielsoins (e.g., cannabielsoic acid B (CBEA-B), cannabielsoin (CBE), and cannabielsoin acid A (CBEA-A)), cannabigerols (e.g., cannabigerol (CBG), cannabigerol monomethylether (CBGM), cannabigerolic acid (CBGA), cannabigerolic acid monomethylether (CBGAM), cannabigerovarin (CBGV), and cannabigerovarinic acid (CBGVA)), cannabinols and cannabinodiols (e.g., cannabinodiol (CBND), cannabinodivarin (CBVD), cannabinol (CBN), cannabinol methylether (CBNM), cannabinol-C2 (CBN-C2), cannabinol-C4 (CBN-C4), cannabinolic acid (CBNA), cannabiorcool (CBN-C1), and cannabivarin (CBV)), cannabitriols (e.g., 10-Ethoxy-9-hydroxy-delta-6a-tetrahydrocannabinol, 8,9-Dihydroxy-delta-6a-tetrahydrocannabinol, cannabitriol (CBT), and cannabitriolvarin (CBTV)), delta-8-tetrahydrocannabinols (e.g., delta-8-tetrahydrocannabinol (Δ⁸-THC) and delta-8-tetrahydrocannabinolic acid (Δ⁸-THCA), and delta-9-tetrahydrocannabinols (e.g., delta-9-tetrahydrocannabinol (THC), delta-9-tetrahydrocannabinol-C4 (THC-C4), delta-9-tetrahydrocannabinolic acid A (THCA-A), delta-9-tetrahydrocannabinolic acid B (THCA-B), delta-9-tetrahydrocannabinolic acid-C4 (THCA-C4), delta-9-tetrahydrocannabiorcol (THC-C1), delta-9-tetrahydrocannabiorcolic acid (THCA-C1), delta-9-tetrahydrocannabivarin (THCV), and delta-9-tetrahydrocannabivarinic acid (THCVA)).

Conjugate: As used herein, “conjugate” refers to a reversible or irreversible connection between two or more substances or components. In some embodiments, for example, gold nanoparticles (AuNPs) and/or other plasmonic metal nanoparticles (MNPs) are connected to antibodies and/or to antigen binding portions thereof. In some embodiments, AuNPs and/or other plasmonic metal nanoparticles (MNPs) are conjugated with antibodies and/or to antigen binding portions thereof via one or more linker compounds.

Detect. As used herein, “detect,” “detecting,” or “detection” refers to an act of determining the existence or presence of one or more target analytes (e.g., a cannabinoid molecule) in a sample.

Epitope: As used herein, “epitope” refers to the part of an antigen (e.g., a cannabinoid molecule) to which an antibody and/or an antigen binding portion binds.

Reaction Mixture: As used herein, “reaction mixture” refers a mixture that comprises molecules and/or reagents that can participate in and/or facilitate a given reaction or assay. A reaction mixture is referred to as complete if it contains all reagents necessary to carry out the reaction, and incomplete if it contains only a subset of the necessary reagents. It will be understood by one of skill in the art that reaction components are routinely stored as separate solutions, each containing a subset of the total components, for reasons of convenience, storage stability, or to allow for application-dependent adjustment of the component concentrations, and that reaction components are combined prior to the reaction to create a complete reaction mixture. Furthermore, it will be understood by one of skill in the art that reaction components are packaged separately for commercialization and that useful commercial kits may contain any subset of the reaction or assay components.

Sample: As used herein, “sample” means anything capable of being analyzed by the methods, devices, and/or systems disclosed herein.

Specifically Bind: As used herein, “specifically bind,” in the context of small molecule detection, refers to a state in which substantially only target chemical structures (e.g., target cannabinoid molecules) are sufficiently associated with a corresponding or cognate binding agent (e.g., an antibody, or antigen binding portion thereof), to the exclusion of non-target chemical structures, such that the association between the target chemical structures and the binding agent can be detected.

Subject. As used herein, “subject” refers to an animal, such as a mammalian species (e.g., human, dog, cat) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). In certain embodiments, the subject is a human. In certain embodiments, the subject is a companion animal, including, but not limited to, a dog or a cat. A subject can be a healthy individual, an individual that has or is suspected of having a disease or a predisposition to the disease, or an individual that is in need of therapy or suspected of needing therapy. The terms “individual” or “patient” are intended to be interchangeable with “subject.”

System: As used herein, “system” in the context of analytical instrumentation refers a group of objects and/or devices that form a network for performing a desired objective.

### DETAILED DESCRIPTION

In some aspects, the present disclosure provides sensitive and rapid detection methods and systems with inexpensive optoelectronic readouts that greatly improve the detection limit (e.g., by 8 times) and reduce the detection time (e.g., to <2 hours) compared to enzyme-linked immunosorbent assay (ELISA) platforms. In some embodiments, the multivalent small molecule sensors disclosed herein were designed by conjugating nano-binders targeting non-competing epitopes on the same molecules onto gold nanoparticles (AuNP) or other MNPs. Such MNP sensors aggregate through binding at the presence of the target small molecule (e.g., cannabinoid molecules), and then precipitate, resulting in increased solution transparency. The solution color change is correlated with small molecule concentration and can be further quantified using a spectrometer or a portable an electronic readout system comprising light emitting diode (LED), photodiode, and battery. As described further herein, using cannabidiol (CBD) as the target, we demonstrate optoelectronic detection<100 pM in urine and saliva with a high specificity (distinguishing from closely related tetrahydrocannabinol (THC)) and large dynamic range (5 logs). These cost-effective, portable and accurate systems can be advantageously implemented in a wide variety of applications, including biomarker diagnostics, drug screening, and drug discovery.

Embodiments of the present disclosure provide plasmonic metal nanoparticle (MNP) based colorimetric assays to identify and quantify cannabinoid molecules using optical and electronic readouts. Analytes (e.g., targeted cannabinoid molecules) modulate the extent of MNP clustering and precipitation, and accordingly, changes the suspension color and intensity, which can be quantified to determine the concentration, binding affinity, and even binding epitope of the analyte. This disclosure has the capability to substantially promote the availability of drug screening and monitoring tests.

Some embodiments of the present disclosure provide a portable colorimetric sensor design for rapid detection of cannabinoid molecules. In some embodiments, different assay variants can be used, including MNP in suspension and dried states (bare-eye readout), spectroscopic quantification, and optical and structural analysis. In some embodiments, the MNP shape and size, analyte and MNP concentration, and binding affinity affect the limit of detection, dynamic range, and assay time will be incorporated into the assays of the present disclosure. Additionally, antibodies, or antigen binding portions thereof, (e.g., monoclonal antibodies, nanobodies, and/or the like) that bind to epitopes of targeted cannabinoid molecules can be conjugated on MNPs of different geometries and materials that display distinct colors. Such heterogeneous MNPs can be used to establish a sandwich-type assay capable of detecting multiple types of antigens by bare eyes. As would be recognized by one of ordinary skill in the art based on the present disclosure, the compositions, reaction mixtures, kits, devices, assays, and systems described herein can be used with any small molecule, such as cannabinoid molecules in a given sample recognized by antibodies, or antigen binding portions thereof, conjugated with the MNPs.

Embodiments of the present disclosure also include a new plasmonic metal nanoparticle (MNP) based colorimetric assay platform that will support a variety of sensing schemes, including multiplexed detection of cannabinoid molecules. Using different assay variants, such as MNP in suspension (e.g., in microcentrifuge tubes or customized PDMS well plate) and dried states (e.g., on glass or gold surface), structural analysis and optical detection are combined with intuitive physical pictures and a theoretical mathematical model to comprehensively understand the mechanisms of MNP-based multivalent analyte-binding. Such studies provide a foundation to further incorporate heterogeneous MNPs displaying distinct colors from blue to red to improve specificity, achieve multiplexed detection, and expand assay functionalities. In addition, portable and inexpensive detecting instruments as disclosed herein provide more precise quantification than bare-eye readout, feasible for clinical settings and field deployment in some embodiments.

In some embodiments, the assays disclosed herein can deliver accurate detection results in about 20 minutes or less by accelerating AuNP and/or other MNP crosslinking, for example, using centrifuge concentration. Multiple characterization methods, including scanning electron microscopy (SEM) and dark field scattering imaging, among other techniques, can be applied for quantitative analysis in cannabinoid molecule detection with less than one nM sensitivity and accuracy. In some embodiments, the present disclosure also demonstrates the feasibility of detecting cannabinoid molecules in saliva, urine, serum, or other sample types with miniaturized portable UV-visible spectrometers for point-of-care detection. In some embodiments, for example, the MNP solution-based colorimetric assays and other aspects disclosed herein provide ultra-high sensitivity, low cost and electricity free colorimetric detection, which can be readily utilized for point-of-care detection of target cannabinoid molecules. These and other aspects will be apparent upon a complete review of the present disclosure, including the accompanying figures.

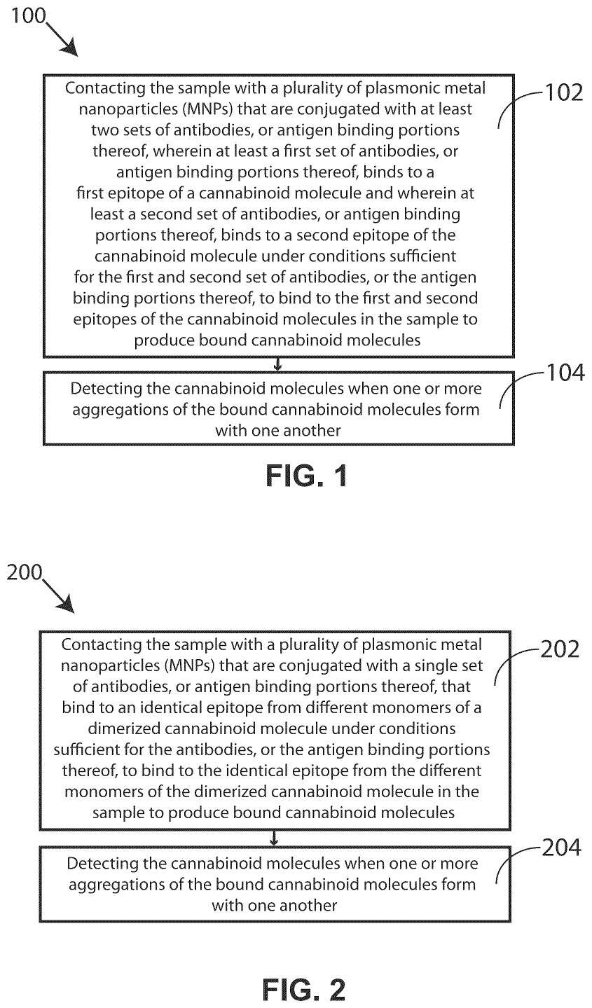

To illustrate, FIG. 1 is a flow chart that schematically shows exemplary method steps of detecting cannabinoid molecules in a sample according to some embodiments. As shown, method 100 includes contacting the sample with a plurality of plasmonic metal nanoparticles (MNPs) (e.g., gold nanoparticles (AuNPs) or the like) that are conjugated with at least two sets of antibodies (or antigen binding portions thereof, (e.g., monoclonal antibodies, nanobodies, etc.)) in which a first set of antibodies (or antigen binding portions thereof) binds to a first epitope of a cannabinoid molecule and in which a second set of antibodies (or antigen binding portions thereof) binds to a second epitope of the cannabinoid molecule under conditions sufficient for the first and second set of antibodies (or the antigen binding portions thereof) to bind to the first and second epitopes of the cannabinoid molecule in the sample to produce bound cannabinoid molecules (step 102).

In another embodiment, FIG. 2 provides a flow chart that schematically shows other exemplary method steps of detecting cannabinoid molecules. As shown, step 202 of method 200 includes contacting the sample with a plurality of MNPs (e.g., gold nanoparticles (AuNPs) or the like) that are conjugated with a single set of antibodies (or antigen binding portions thereof (e.g., monoclonal antibodies, nanobodies, etc.)) that bind to an identical epitope from different monomers of a dimerized cannabinoid molecule under conditions sufficient for the antibodies (or the antigen binding portions thereof) to bind to the identical epitope from the different monomers of the dimerized cannabinoid molecules in the sample to produce bound cannabinoid molecules. Essentially any sample type is used in performing method 100 or method 200. In some embodiments, for example, the sample comprises blood, plasma, or serum. In some embodiments, the sample comprises saliva or sputum. In some embodiments, the sample comprises urine. In some embodiments, a given antibody, or antigen binding portions thereof, comprises a mass of between about 10 kDa and about 20 kDa (e.g., about 11 kDa, about 12 kDa, about 13 kDa, about 14 kDa, about 15 kDa, about 16 kDa, about 17 kDa, about 18 kDa, or about 19 kDa). In some embodiments, a given antibody, or antigen binding portions thereof, comprises an equilibrium dissociation constant (K_{D}) on the order of between about 1 nM and about 100 nM (e.g., about 5 nM, about 10 nM, about 15 nM, about 20 nM, about 30 nM, about 40 nM, about 50 nM, about 60 nM, about 70 nM, about 80 nM, or about 90 nM). The production of antibodies and antigen binding portions thereof suitable for use with methods, devices, and other aspects of the present disclosure are described further herein or otherwise know to a person having ordinary skill in the art.

Methods 100 and 200 each also include detecting the cannabinoid molecules when aggregations of the bound cannabinoid molecules form with one another to thereby detect the cannabinoid molecules in the sample (step 104 or 204). In some embodiments, the detection step includes determining a change in absorbance at a resonance wavelength of the MNPs. In some embodiments, method 100 or 200 includes quantifying an amount of the cannabinoid molecules in the sample. In some embodiments, method 100 or 200 further includes centrifuging the aggregations of the bound cannabinoid molecules prior to and/or during the detecting step. In some embodiments, method 100 or 200 further includes freezing the aggregations of the bound cannabinoid molecules prior to the detecting step. In some embodiments, method 100 or 200 includes drop casting the aggregations of the bound cannabinoid molecules prior to the detecting step. In some embodiments, method 100 or 200 includes obtaining the sample from a subject. In some embodiments, method 100 or 200 includes administering one or more therapies to the subject when a targeted cannabinoid molecule is detected in the sample. In some embodiments, method 100 or 200 includes detecting the cannabinoid molecules within about 20 minutes or less of obtaining the sample from the subject. The method 100 or 200 may detect the cannabinoid molecules within 25 minutes, 24 minutes, 23 minutes, 22 minutes, 21 minutes, 20 minutes, 18 minutes, 16 minutes, 14 minutes, 12 minutes, 10 minutes, 8 minutes, 6 minutes, 5 minutes, 4 minutes, 3 minutes, 2 minutes 1 minute, or any range between these values. In some embodiments, method 200 includes repeating the method using one or more longitudinal samples obtained from the subject (e.g., to monitor the subject's cannabis usage over time). In some embodiments, the detecting step comprises measuring a colorimetric change when the one or more aggregations of the bound cannabinoid molecules form with one another. In some embodiments, method 100 or 200 includes visually detecting the colorimetric change when the one or more aggregations of the bound cannabinoid molecules form with one another. In some embodiments, method 100 or 200 includes detecting the colorimetric change when the one or more aggregations of the bound cannabinoid molecules form with one another using a spectrometer. In some embodiments, a concentration of cannabinoid molecules in the sample is about 15 nM or less (e.g., when visually detecting the colorimetric change). In some embodiments, a concentration of cannabinoid molecules in the sample is about 100 pM or less (e.g., when detecting the colorimetric change using a spectrometer). In some embodiments, the MNPs (e.g., AuNPs and/or the like) comprise a substantially spherical shape. In some embodiments, the MNPs comprise a cross-sectional dimension of between about 10 nm and about 100 nm.

Some aspects of the present disclosure include a colorimetric sensing mechanism that uses nanobody-coated metal nanoparticles for cannabinoid detection. Some of these embodiments include synthesizing nanobodies for a targeted cannabinoid molecule. Nanoparticles are optionally prepared by producing the nanobodies in bacterial host cells, biotinylating the nanobodies, and coating AuNPs with the biotinylated nanobodies. Aggregations of bound cannabinoid molecules are then detected when samples are contacted with the nanobody coated AuNPs in some embodiments. Some embodiments include the use of a portable readout system, which includes a quantitative electronic readout with light-emitting diodes (LEDs) and photodetectors, bare-eye colorimetric readout, and a quantitative spectroscopic analysis using a polydimethylsiloxane (PDMS) well plate and a spectrometric detector.

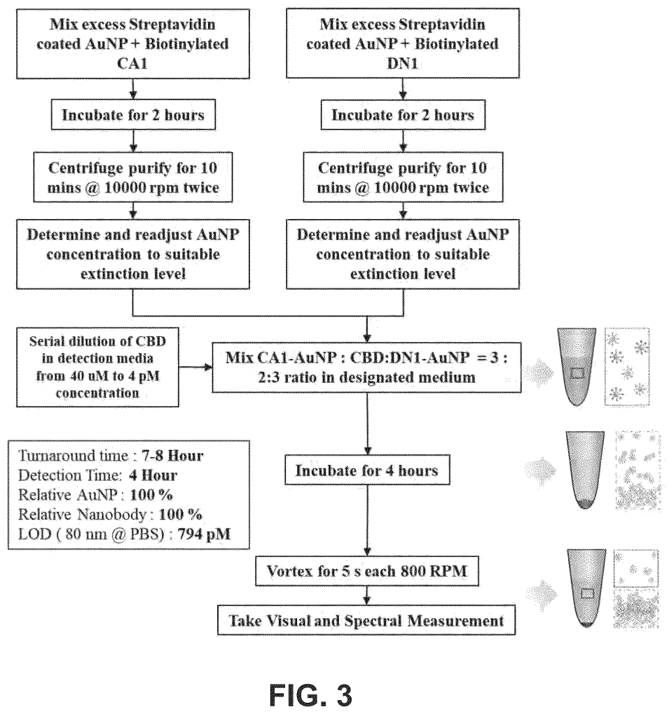

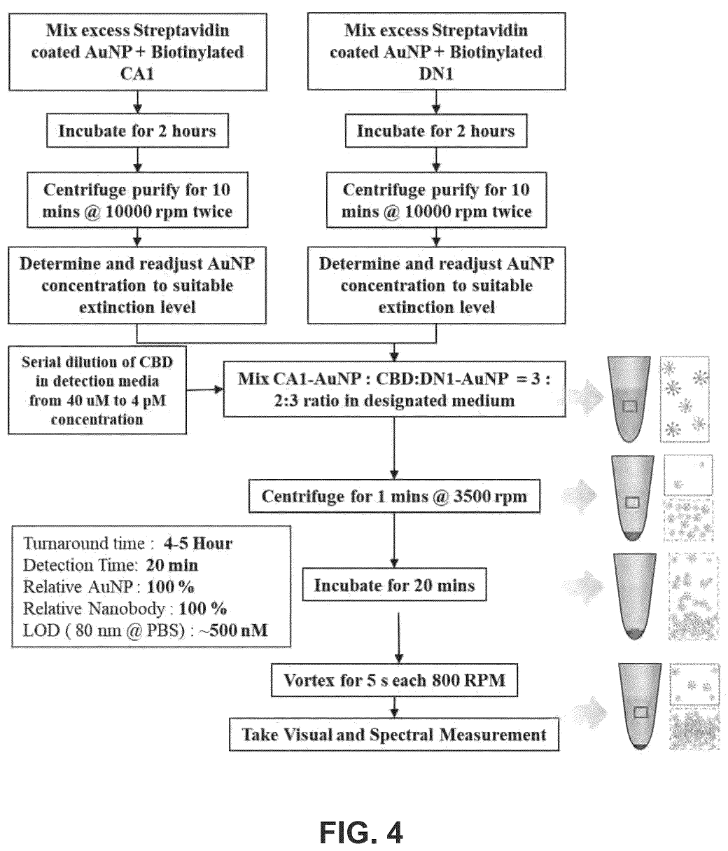

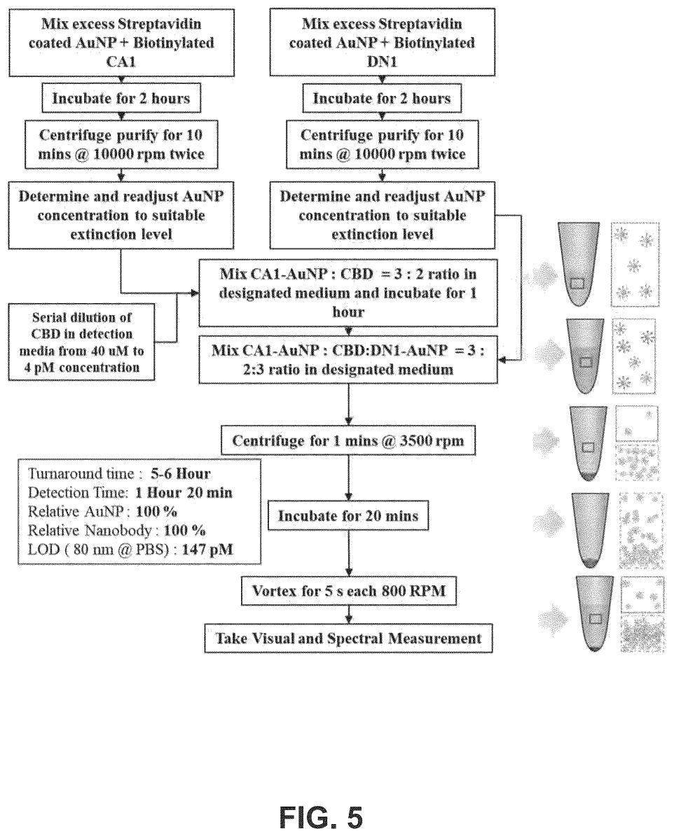

To further illustrate, FIGS. 3-5 show schematic diagrams of exemplary work flows for CBD detection assays. More specifically, FIG. 3 is a schematic diagram of a work flow of an incubation-based CBD detection assay according to one exemplary embodiment. As shown, the method includes separately mixing excess streptavidin coated AuNPs with a biotinylated CBD anchor binder (CA) and a biotinylated CBD dimerization binder (DN). Those mixtures are then each incubated and centrifuge purified. The AuNP concentration of each mixture is then determined and readjusted to suitable extinction levels. Those mixtures are then combined with one another and the CBD sample at a selected ratio in a designated medium. In this embodiment, visual and spectral measurements of the mixture are made after being incubated and vortexed. In contrast, FIG. 4 is a schematic diagram of a work flow of a centrifugation enhanced CBD detection assay according to one exemplary embodiment. As shown, the steps to form the mixture CA-AuNP, DN-AuNP, and sample are the same as those depicted in FIG. 3. However, prior to taking visual and spectral measurements of the mixture, the process shown in FIG. 4 includes a centrifugation step in addition to incubation and vortexing steps. As a further illustration, FIG. 5 is a schematic diagram of a work flow of a pre-incubated centrifuge enhanced CBD detection assay according to one exemplary embodiment. The exemplary process shown in FIG. 5 is the same as that depicted in FIG. 4, except that the CBD sample is first mixed with CA-AuNP mixture at a selected ratio in a designated medium and incubated prior to being combined with the DN-AuNP mixture at a selected ratio.

In some embodiments, the present disclosure provides a composition that comprises a plurality of plasmonic metal nanoparticles (MNPs) that are (i) conjugated with at least two sets of antibodies, or antigen binding portions thereof, in which at least a first set of antibodies, or antigen binding portions thereof, binds to a first epitope of a targeted cannabinoid molecule protein and which at least a second set of antibodies, or antigen binding portions thereof, binds to a second epitope of the targeted cannabinoid molecule in the sample; or (ii) conjugated with a single set of antibodies, or antigen binding portions thereof, that bind to an identical epitope from different monomers of a dimerized cannabinoid molecule in the sample.

In another aspect, the present disclosure provides a reaction mixture that includes a sample comprising a targeted cannabinoid molecule, and a plurality of MNPs (e.g., gold nanoparticles (AuNPs)) that are (i) conjugated with at least two sets of antibodies, or antigen binding portions thereof, (e.g., nanobodies, etc.) wherein at least a first set of antibodies, or antigen binding portions thereof, binds to a first epitope of a cannabinoid molecule and wherein at least a second set of antibodies, or antigen binding portions thereof, binds to a second epitope of a cannabinoid molecule in the sample; or (ii) conjugated with a single set of antibodies, or antigen binding portions thereof, that bind to an identical epitope from different monomers of a dimerized cannabinoid molecule in the sample.

In another aspect, the present disclosure provides a device that includes at least one reaction chamber (e.g., a body structure comprising an array of wells or the like) or substrate comprising a plurality of MNPs (e.g., gold nanoparticles (AuNPs) and/or other MNPs) that are conjugated with at least two sets of antibodies, or antigen binding portions thereof, wherein at least a first set of antibodies, or antigen binding portions thereof, binds to a first epitope of a cannabinoid molecule and wherein at least a second set of antibodies, or antigen binding portions thereof, binds to a second epitope of a cannabinoid molecule when the reaction chamber or substrate receives a sample that comprises the cannabinoid molecule under conditions sufficient for the first and second set of antibodies, or the antigen binding portions thereof, to bind to the first and second epitopes of the cannabinoid molecules in the sample to produce bound cannabinoid molecules and one or more aggregations of the bound cannabinoid molecules to produce a colorimetric change in the reaction chamber; or (ii) conjugated with a single set of antibodies, or antigen binding portions thereof, that bind to an identical epitope from different monomers of a dimerized cannabinoid molecule under conditions sufficient for the antibodies, or the antigen binding portions thereof, to bind to the identical epitope from the different monomers of the dimerized cannabinoid molecule in the sample to produce bound cannabinoid molecules and one or more aggregations of the bound cannabinoid molecules to produce a colorimetric change in the reaction chamber. In some embodiments, the one or more aggregations of the bound cannabinoid molecules are drop cast in or on the reaction chamber or substrate. In other embodiments, the one or more aggregations of the bound cannabinoid molecules may be deposited in or on the reaction chamber or substrate in a variety of deposition techniques. The deposition technique may be spin coating, dip coating, spray coating or any other similar technique known to one of skill in the art. In some embodiments, a kit includes the device.

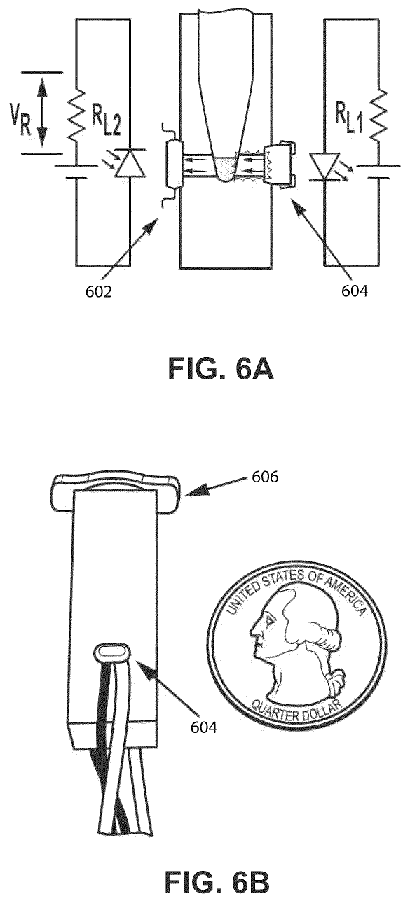

In another aspect, the present disclosure provides a system that includes a device, comprising at least one reaction chamber or substrate comprising a plurality of gold nanoparticles (AuNPs) and/or other MNPs (e.g., MNPs that comprise silver, copper, aluminum, platinum, palladium, or the like) that are (i) conjugated with at least two sets of antibodies, or antigen binding portions thereof, wherein at least a first set of antibodies, or antigen binding portions thereof, binds to a first epitope of a cannabinoid molecule and wherein at least a second set of antibodies, or antigen binding portions thereof, binds to a second epitope of a cannabinoid molecule when the reaction chamber receives a sample that comprises the cannabinoid molecule under conditions sufficient for the first and second set of antibodies, or the antigen binding portions thereof, to bind to the first and second epitopes of the cannabinoid molecules in the sample to produce bound cannabinoid molecules; or (ii) conjugated with a single set of antibodies, or antigen binding portions thereof, that bind to an identical epitope from different monomers of a dimerized cannabinoid molecule when the reaction chamber receives a sample that comprises the cannabinoid molecule under conditions sufficient for the antibodies, or the antigen binding portions thereof, to bind to the identical epitope from the different monomers of the dimerized cannabinoid molecule in the sample to produce bound cannabinoid molecules. The system also includes an electromagnetic radiation detection apparatus positioned, or positionable, within sufficient proximity to the device to detect one or more colorimetric changes produced in or on the reaction chamber or substrate when one or more aggregations of the bound cannabinoid molecules form with one another in or on the reaction chamber or substrate. In some embodiments, the electromagnetic radiation detection apparatus comprises a spectrometer. In some embodiments, the electromagnetic radiation detection apparatus comprises a microscope. As a further illustration, FIGS. 6A and 6B schematically show aspects of an electronic detection system that comprises an LED circuit 604, a photodiode circuit 602, and a 3D printed holder for an Eppendorf tube 606. Additional exemplary devices and systems are described further herein.

### Example

### Optoelectronic Small Molecule Detection by Coupling Chemically Induced Dimerization to Metal Nanoparticles

### Results and Discussion

### CBD CID System Selection

We developed a combinatorial binders-enabled selection of CID (COMBINES-CID) method for small molecule detection (see also, Kang, Shoukai, et al. “COMBINES-CID: An efficient method for de novo engineering of highly specific chemically induced protein dimerization systems,” Journal of the American Chemical Society 141.28 (2019): 10948-10952 and Patent Publication No. US 2022/0177605 A1). The CBD CID system was isolated from a combinatorial nanobody library of over 109 complementarity-determining region (CDR) peptide sequences. To obtain CBD anchor binder (CA), six rounds of selection were performed using biotinylated CBD as bait, eventually obtaining three unique clones with high specificity. We chose CA14, with the highest protein yield as bait to do dimerization binder (DB) selection. 24 unique DBs were identified after four rounds of biopanning. The two most stable CBD CID systems, CA14-DB21 (Kd=56 nM) and CA14-DB18 (Kd=560 nM), were expressed as a C-terminal Avi-tagged and His-tagged form in E. coli, purified by Ni-affinity and biotinylated by BirA and then site-specifically conjugated to gold nanoparticles as previously reported.

### Preparation of Multivalent Small Molecule Sensor

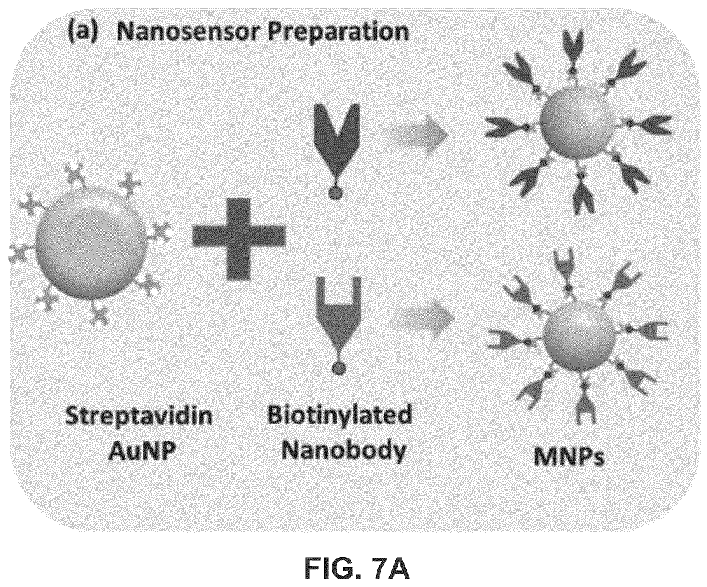

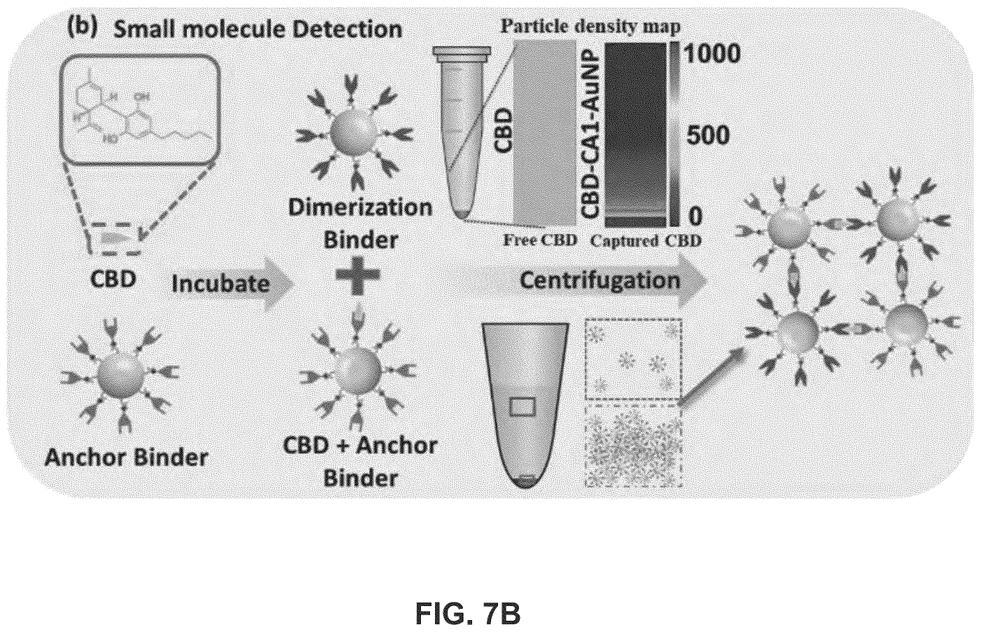

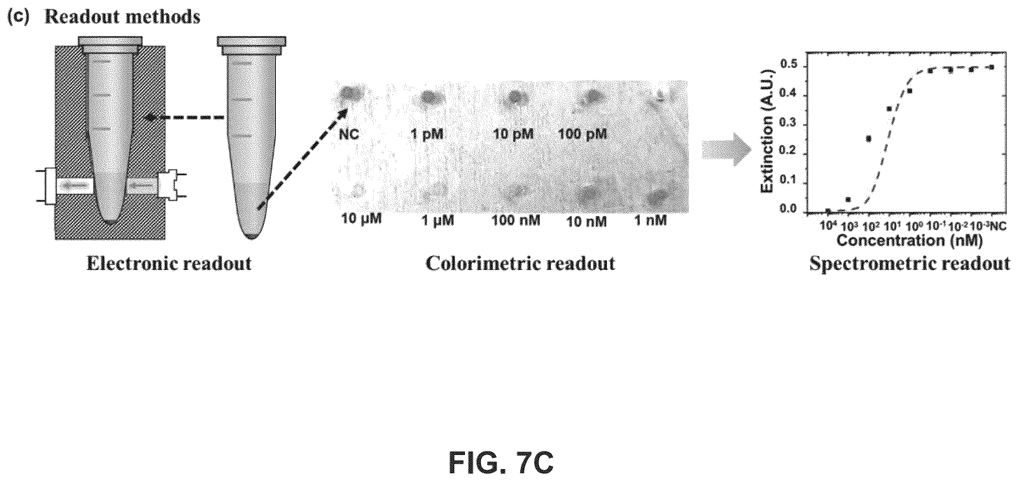

In this small molecule assay, we utilized AuNPs of 40 to 100 nm covalently coated with high-density streptavidin proteins (up to 1280 for 80 nm AuNPs) for sensing, conceptually similar to our recently reported antigen sensors. Upon mixing with biotinylated nanobodies (anchor binder CA14 and dimerization binders DB18 or DB21), these AuNPs formed multivalent sensors (FIG. 7A). Upon the introduction of small molecules, these multivalent in-solution sensors display improved effective affinity, beneficial for accelerated molecular binding and higher sensitivity compared to monovalent detection systems. The molecular recognition is accompanied by aggregation of the AuNP sensors, resulting in sedimentation. This sedimentation-based detection mechanism differs fundamentally from other plasmonic colorimetric assays, where the detection is based on resonance wavelength shift caused by the formation of dimers and oligomers of small AuNP (5-20 nm) still present in the solution. First, the larger AuNPs used by us have larger surface areas, thus capable of hosting more nanobodies and presenting a high binding affinity for high-quality multivalent molecular recognition. Further, the larger NP sizes also make them more responsive to centrifugation-induced precipitation (FIG. 7B), which is important to shorten the assay time and improve the detection limit (e.g. 2 hours, compared to 5 hours by incubation only). The AuNPs reliably display molecular concentration-dependent modulation in extinction signals in visible wavelengths. Unlike detecting resonance wavelength shift, which demands bulky and expensive spectrometer for high-precision peak identification, the detection of extinction intensity modulation can also be accurately performed by simple LEDs and photodetectors at the target wavelengths only without analyzing the complete spectra (FIG. 7C), thus enabling portable, rapid, inexpensive and digital diagnostics.

### Pre-Incubation and Centrifugation Enhanced Detection

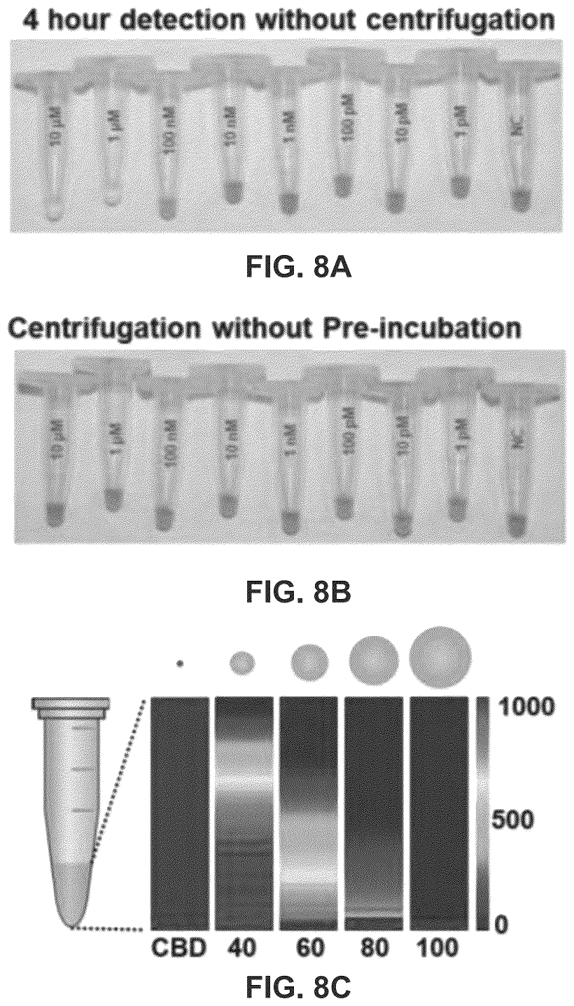

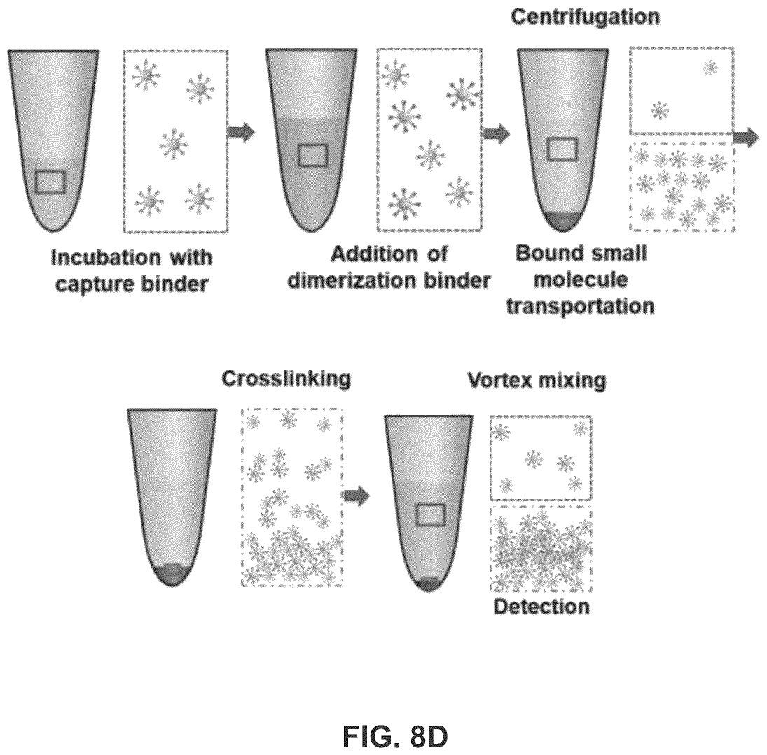

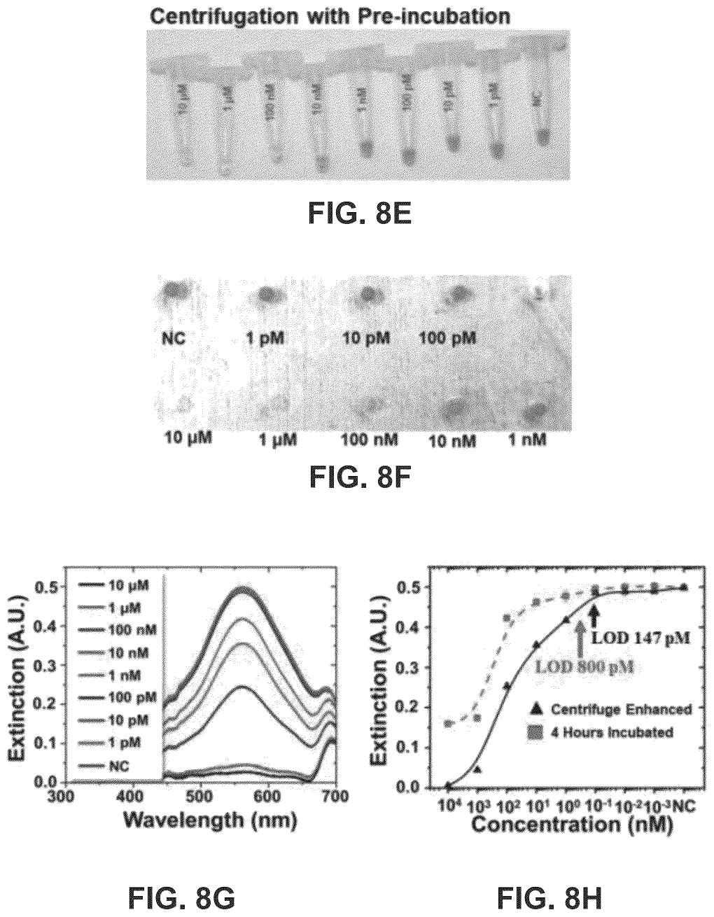

As discovered in our previous work, the sample preparation methods, particularly incubation and centrifugation, have a significant impact on sensing accuracy and detection time of the AuNP sensors. Here, we have compared two methods for CBD detection, i.e. incubation only and incubation followed with centrifugation before detection (“IC-detect”). For the incubation-only approach, 80 nm AuNPs coated with purified CA14 and DB21 were incubated with the CBD molecules for 4 hours (FIG. 8A). Then, the top 5 μL liquid was pipette-loaded in a custom-made PDMS plate reader and spectroscopically measured using a UV-visible spectrometer coupled to an upright microscope. A significant color contrast was observed between 100 nM CBD and the NC (control sample with no CBD) sample (FIG. 8A). To quantify the CBD detection, we further extracted the extinction intensity at the peak wavelength (˜560 nm) for 80 nm AuNPs and plotted it against CBD concentration. The incubation-based sensing yielded a LOD of ˜0.7 nM (FIG. 8H), comparable to the sandwich ELISA based detection method applied previously with the same set of co-binding system. The incubation-based system proved the feasibility of detecting small molecules such as CBD with a detection time similar to ELISA (typically around 5 hours) but much faster than traditional MS systems.

To further improve the assay sensitivity and reduce the assay time, we briefly studied the impact of AuNP sedimentation process on sensing by theoretical analysis. In such AuNP-based sensing system, two critical parameters governing the assay time and performance are aggregation time constant τₐ and sedimentation time constant τₛ. In determining τₐ, we employed a simplified version of Smoluchowski's coagulation equation to evaluate the empirical parameter P, defined as the probability of a collision resulting in a binding event, and found the best fit was ˜1. This indicates that the multivalence of the conjugated AuNPs increases the potential binding affinity compared to mono-binding process, significantly different from traditional surface-based detection such as ELISA. This also implies that ELISA-determined binding constants are non-ideal to precisely predict the effective affinity observed in our solution-based multivalent system. Additionally, τₐ can also be reduced significantly by increasing the AuNP concentration, which could be achieved by applying centrifugation to localize the AuNPs at the tube bottom. Further, we calculated τₛ=z/(s·g) using Mason-Weaver equation, where z is the precipitation path (for example, the height of colloid liquid), g is the gravitation constant, and s is the sedimentation coefficient dependent on the physical properties of AuNPs and buffers. For a liquid height of z˜3.5 mm (approximated for 16 μL liquid in a 0.5 ml Eppendorf tube), we found the τₛ reduced from 26 hours to 20 mins for 80 nm AuNP 800 nm clusters, respectively. This highlights that larger clusters formed during CBD-nanobody binding will precipitate rather quickly. Assisted by simulation, we can also visualize that 80 nm AuNPs have a very narrow equilibrium gradient distribution in micro-molar concentration, more specifically that most of the dimers and trimers are at the tube bottom. Therefore, only floating monomers modulate the observed optical extinction intensity, while the AuNP dimers or oligomers would not contribute to the solution signals.

The theoretical analysis points out that increasing the AuNP concentration and decreasing the liquid height, i.e. volume, would enable us to detect CBD faster. However, too high AuNP concentration would have resulted in saturation of optical extinction at lower CBD concentration, with a negative outcome of higher noise and lower detectability. On the other hand, the too small liquid volume could bring challenges to separate the precipitates from the top liquid and complicate signal transduction. Here, we applied the IC-Detect approach similar to the method we employed before in rapid and sensitive Ebola and SARS-COV-2 antigen sensing. However, the incubation time (1 to 20 min) for protein sensing was found insufficient here to produce a visible color change except at the highest (10 μM) CBD concentration (FIG. 8B). To investigate this phenomenon, we used Stokes centrifugal force equation to calculate the spatial profile, particle density (FIG. 8C) under centrifugal force for CBD and AuNPs of different sizes from 40 to 100 nm. The simulation shows this centrifugation step, although effectively concentrating the AuNPs at the bottom of the tube within 2 mins, is incapable of concentrating CBD. Still broadly distributed within the tubes, the small CBD molecules at the tube bottom are limited, and therefore the CBD-induced AuNP clustering process became slow and CBD diffusion-limited. To circumvent this slow reaction issue, we opted to use the higher affinity binder CA14 (6 μM compared to 56 μM for DB21) as a CBD carrier to transport target CBD molecules to the reaction zone at the tube bottom. More specifically, we pre-incubated the CA14 coated AuNPs with CBD for 2 hours, followed by mixing with the DB21 coated AuNPs and centrifugation (FIG. 8D). This nanobody/AuNP-mediated pre-incubation of CBD (FIG. 8E) evidently improved the detection of CBD (FIG. 8E). The concentration-dependent CBD sensing results were further quantified by extracting the top 5 μL in a PDMS plate (FIG. 8F), examining their optical extinction (FIG. 8G) and recording the extinction peaking values at the AuNP resonance (560 nm) (FIG. 8H). Clearly, the incubation-based (dashed line, FIG. 8H) and IC-detect methods (solid black line, FIG. 8H) display similar concentration-dependent sensing curves; however, the LOD for the IC-detect method (˜144 pM) is about 5 times better than the incubation-based detection (˜700 pM) and the traditional Sandwich ELISA with the same reagents (˜800 pM).

### Size, Structural and Particle Tracking Analysis

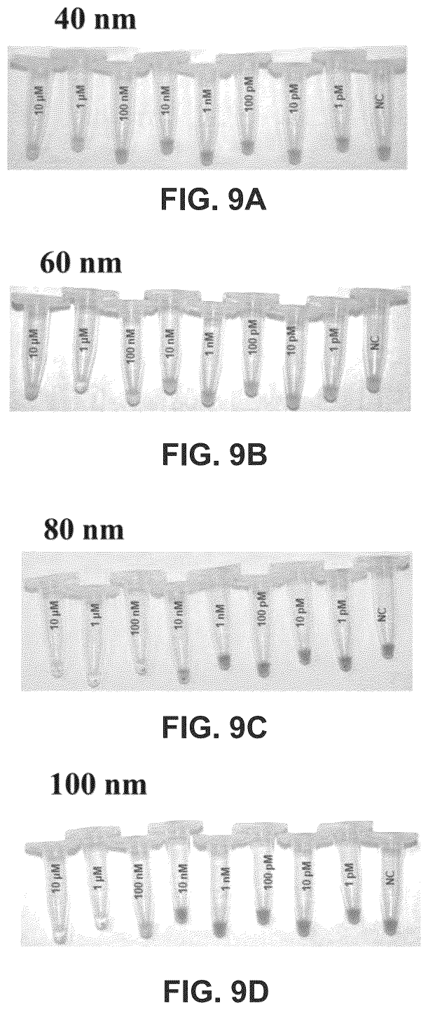

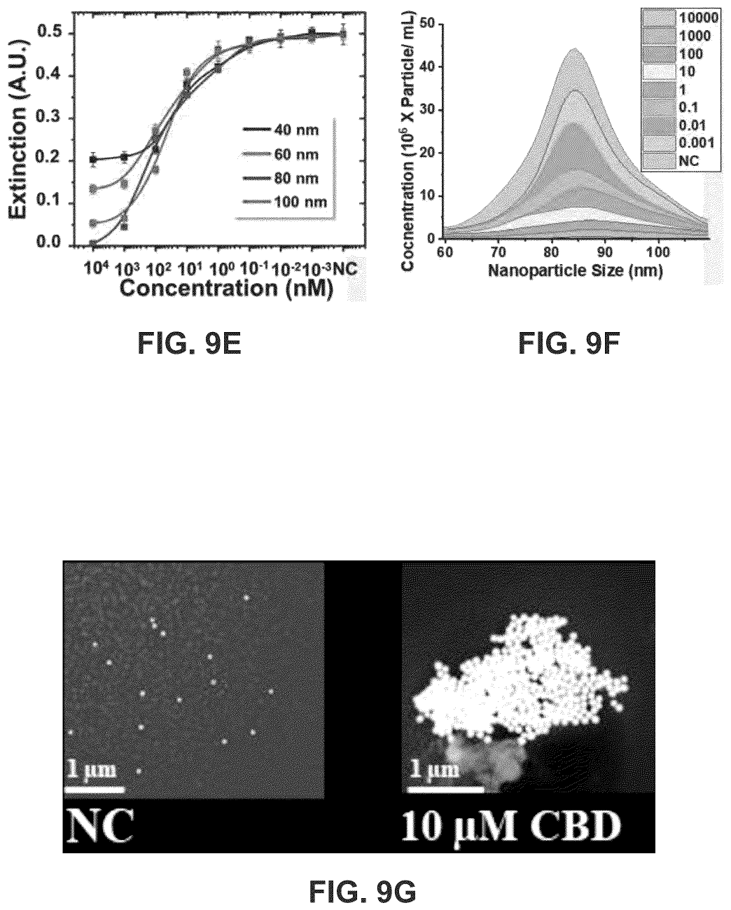

To analyze the effect nanoparticle size plays in CBD detection, we employed the IC-detect assay for CBD with different AuNP sizes (40, 60, 80 and 100 nm) (FIGS. 9A-9D) similar to our previous study, all the AuNPs could easily differentiate the color of 1 μM CBD concentration from the NC sample. However, further analysis of the extinction signals showed that 80 and 100 nm AuNPs performed the best of dynamic range and LOD (FIG. 9E). The underlying mechanism involves multidisciplinary studies of analyte-ligand binding, plasmonic AuNP effects, and AuNP aggregation and sedimentation behavior. The effective concentration [NP]ₑ of a multivalent nanoparticle is correlated with the concentration of the nanoparticle [NP] and the amount of surface ligands [S] as [NP]ₑ∝[NP][S]. The AuNP localized surface plasmon resonance (LSPR) extinction is dependent on its concentration [NP] and diameter d following σₑₓₜ∝[NP]d³, and therefore the AuNP concentration in our test was lower at larger sizes following

$$[ NP ] ∝ \frac{1}{{d}^{3}}$$

considering equal extinctions. Further considering [S] is also related to the surface area and hence diameter of the particles as [S]∝d², we obtain [NP]ₑ∝d. As discussed previously, a higher [NP]ₑ, or small AuNPs at equal optical extinction, is favored to promote reaction and hence the AuNP aggregation. Yet, sedimentation time also increases with smaller AuNPs. This tradeoff is unavoidable for the incubation-based sensing method. In comparison, for IC-detect method, the use of centrifugation effectively concentrates the AuNPs to promote faster reaction even at larger particle sizes. For example, we have performed Stoke's centrifugal simulation shows that 2-min centrifugation at 1200 g could condense 80 nm AuNPs by a factor of ˜50 but only condense the 40 nm AuNPs by a factor of ˜10. In other words, this means the occupied volume by smaller nanoparticles after centrifugation was a few times larger, diminishing their concentration advantage (when designed at the same optical density, or comparable extinction). Further, given a slower sedimentation rate, the smaller nanoparticles behaved less effective in promoting faster reactions. On the other hand, because only mild centrifugal force was applied to minimize non-specific physical aggregation, nanoparticles larger than 80 nm after centrifugation still had lower concentration compared to 80 nm sizes, thus also less efficient in sensing performance.

As a result of the above analysis, we chose 80 nm AuNPs for CBD sensing in this work. In order to better verify the working mechanism, we tested CBD with 0.036 nM AuNPs, extracted the top-level liquid, and diluted for nanoparticle tracking analysis (NTA) measurement (FIG. 9F). From the NC sample, the AuNP size distribution (average 80 nm, 3-sigma deviation 20 nm) is consistent with expectation, considering the coating of streptavidin and nanobodies on the AuNPs. Such NTA analysis serves to verify that the top liquid only contains monomers of the functionalized AuNPs. In addition, we also extracted the bottom 2 μL liquid of all the samples and imaged by transmission electron microscopy (TEM) (FIG. 8F). Clearly, only monomers were observed for the NC sample, evidently showing minimal non-specific AuNP clustering, but AuNP clusters of different sizes were only found for CBD concentrations from 1 pM to 100 nM, providing evidence of three-dimensional AuNP cluster formation in sensing.

### Modular Analytic Model for Centrifugation Enhanced Nanoparticle-Based Antigen Detection Assay

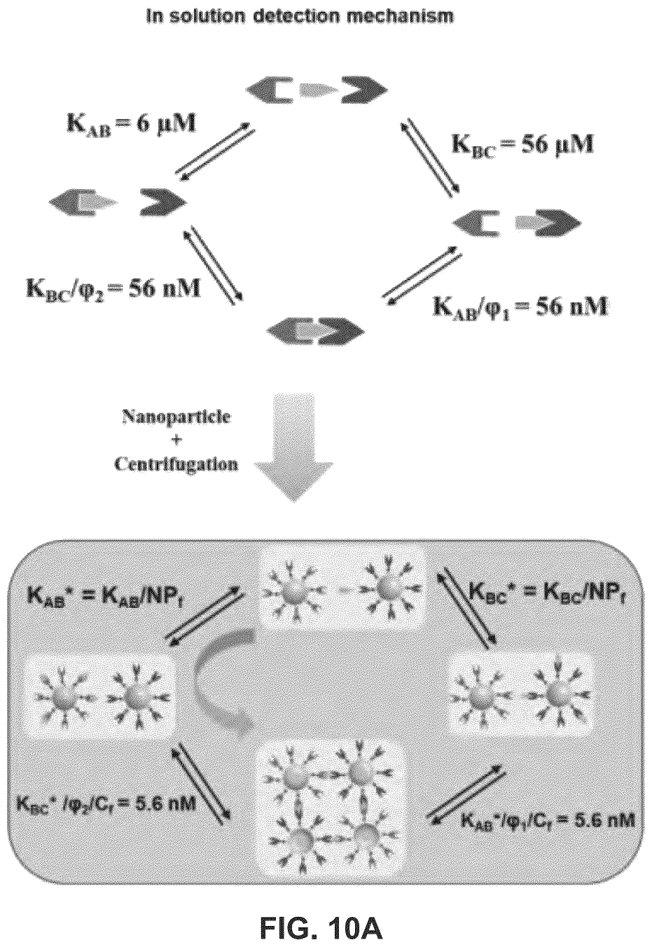

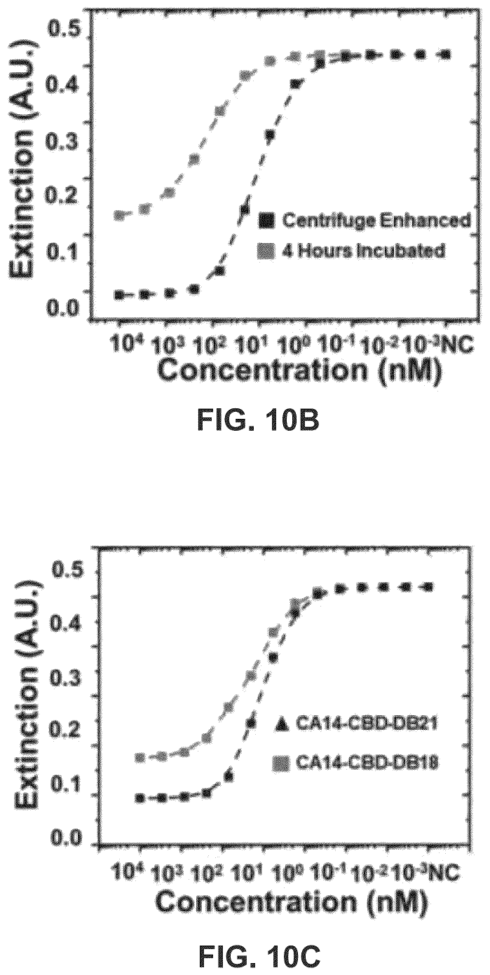

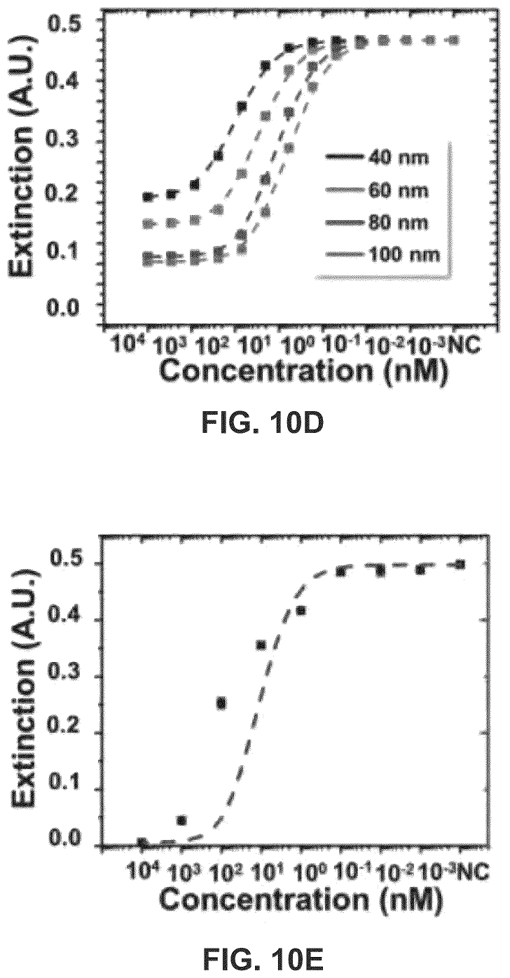

As discussed, a number of factors, including multi-valency recognition sites, sedimentation time, aggregation, etc., can affect the assay preformation. To better understand the complex phenomena, we created a modular analytic model. Briefly, we started with a dynamic model for the reaction kinetics in combination with Smoluchowski's coagulation equation and utilized two different coagulation model (Mason-Weaver equation for gravitational sedimentation and Stokes equation for centrifugal sedimentation) to parameterize the initial conditions as well as to predict the aggregate precipitation. Additionally, we have identified two factors that correlate to the enhancement effect of our system, i.e., NPf signifies the multivalence effect of the nanoparticle sensor (proportional to the surface area or d²) while Cf signifies the nanoparticle concentration effect by centrifugation. In addition, the pre-incubation was introduced to favor the chemical reaction of Capture Nanobody (CA14) and CBD to form the CA14-CBD complex with improved binding affinity amongst the nanotainers. (FIG. 10A). We found that Npf=50 and Cf=100 produced the best experimental fitting for 80 nm AuNP assay. The centrifugation effect (FIG. 10B) is visualized that the incubation-based assay (upper, lighter greyscale curve, Cf=0) had a much worse signal contrast in optical extinction at the highest analyte concentrations (10 μM) compared to that with IC-detect approach (black curve Cf=100), which is similar to what we have observed during experimentation (FIG. 8G). Further, we also simulated the impact of the inherent antigen-antibody binding affinity on the assay performance (FIG. 10C), where we use two different nanobody systems CA14-DB21 (Kd=56 nM) and CA14-DB18 (Kd=560 nM). Interestingly, despite that DB18 has one order of magnitude lower binding affinity, the predicted sensing performance is only slightly worse in the signal contrast, i.e. the optical extinction at the highest concentration is 1.8 compared 0.8 for DB21. This is partially attributed to the fact that DB18 has a higher dissociation constant (560 nM compared to 56 nM), and such high-dissociation is partly compensated with the use of multivalency in-solution AuNPs, which effectively promotes stronger binding Further, the precipitation based readout is much less reversible and more favorable compared to monolayer analyte binding than in a conventional ELISA assay. On the other hand, while the overall effect of nanoparticle size (FIG. 10D) agrees well with our experimental observation (FIG. 9E), experimentally we observed slight worse performance for 100 nm AuNPs, which could be attributed to errors in model parameterization given broader particle size distribution. Lastly, as an example, the fitting of our model for the 80 nm AuNP agrees well with the experimental results (FIG. 10E). These results prove that such an analytical model can be used to guide sensor design and optimization for affinity-based sensing of small molecules, proteins, and probably also nucleic acids. In addition, coupling the modeling with experimental results will also serve to better design and screen the performance of designed synthetic nanobodies.

### Nanobody Affinity Effect and Unfiltered Assay Development

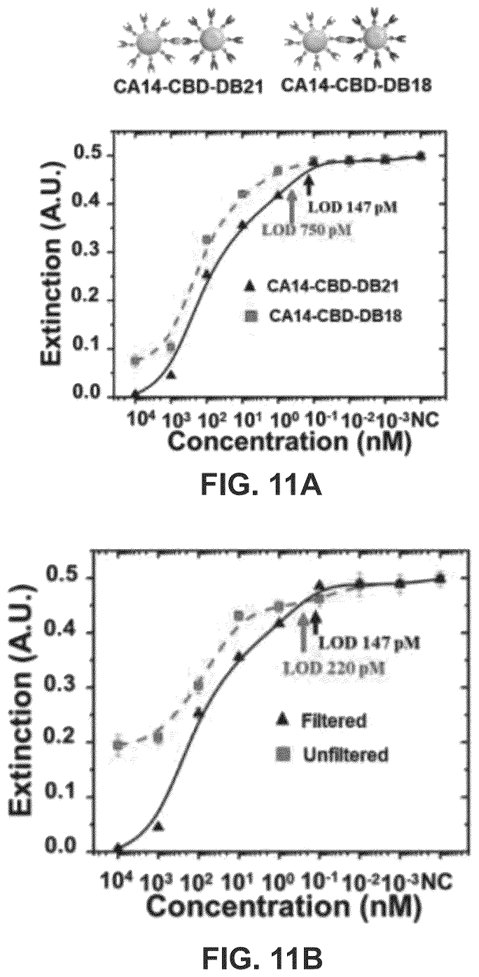

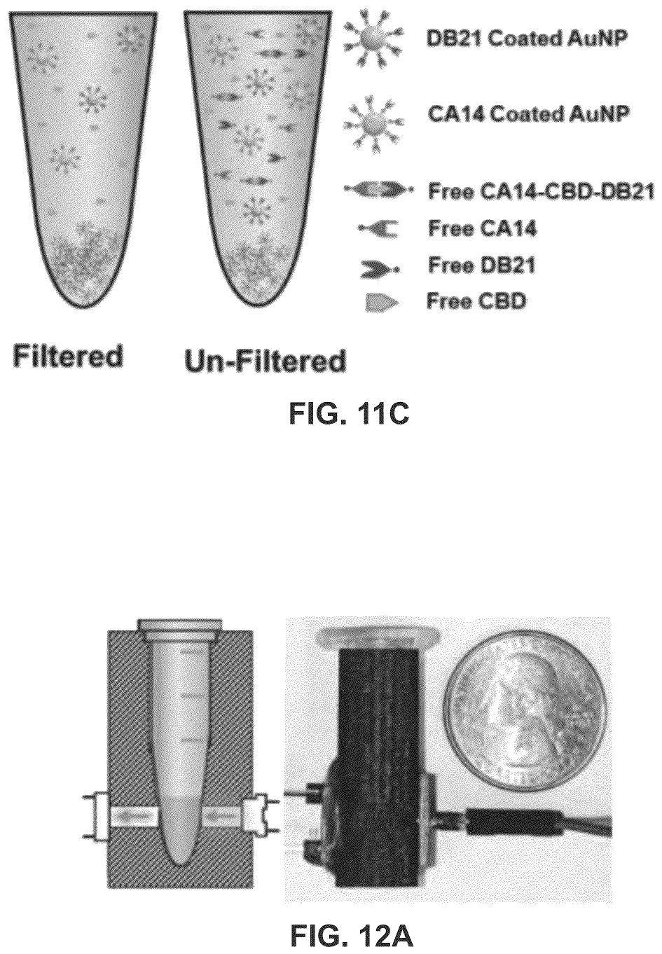

The coupled experimental and modeling analysis proves that our design strategy is successful in small-molecule analyte detection with greatly improved sensitivity compared to ELISA. Yet, it would still require conjugation, purification of the nanobody and the nanoparticle, which would require a centrifuge and training. To explore the feasibility with an even simpler sensing scheme, we tested our system with unfiltered assays, i.e. mixing the nanobody solutions with AuNPs without any post-conjugation purification. In particular, a near stoichiometric ratio (1:1280 for 80 nmAuNP: Nanobody, assumed from the approximate binding site provided by the vendor for 80 nm AuNP) of nanobody and AuNP was mixed and incubated for 30 min, and then our previously discussed IT-detect method was used to detect CBD. This approach not only eliminated the purification process and saved preparation time, but also reduced the AuNP consumption by one third by minimizing purification loss as well as reducing nanobodies consumption by 50- to 100-fold (excess amount was needed for conjugation but then lost during filtration). Despite worse performance (LOD of 750 pM) compared to the filtered assay (LOD˜147 pM, FIG. 11B) due to the presence of partially conjugated AuNPs and free nanobodies (FIG. 11C and Table 1), the LOD is sufficient for most practical applications, particularly in applications where portability, cost, and accessibility are of great importance. Further, optimization, e.g. in the mixing ratio of the AuNPs and nanobody concentrations, could further improve the performance of this unfiltered assay format.

TABLE 1

| | | | | Pre-incubated | Pre-incubated |
|---|---|---|---|---|---|
| | Incubation | | Centrifugation | Centrifuge | Centrifuge |
| Process name/ | with | | Enhanced with | Enhanced with | enhanced without |
| Steps/Performance | Filtration | | Filtration | Filtration | Filtration |
| AuNP:Antibody | 1:2560 | | 1:2650 | 1:2560 | 1:400 |
| 2 Stage Centrifuge | Yes | | Yes | Yes | No |
| Filtration | | | | | |
| 1 Hour Pre-Incubation | No | | No | Yes | Yes |
| with Capture antibody | | | | | |
| Centrifugation | No | | Yes | Yes | Yes |
| Enhanced | | | | | |
| Post Centrifugation | 4 Hour | | 20 min | 20 min | 20 min |
| Incubation Time | (without | | | | |
| | Incubation) | | | | |
| Turn Around time | 7-8 | Hours | 4-5 Hours | 5-6 Hours | 3-4 Hours |
| Detection Time | 4 | Hours | 20 min | 1 Hour 20 min | 1 Hour 20 min |

| | | | | |
|---|---|---|---|---|
| Relative AuNP | 100% | 100% | 100% | 75% |
| consumption | | | | |
| Relative Nanobody | 100% | 100% | 100% | 15% |
| Consumption | | | | |

### Detection of CBD in Urine and Saliva Using Portable Electronic Readout System

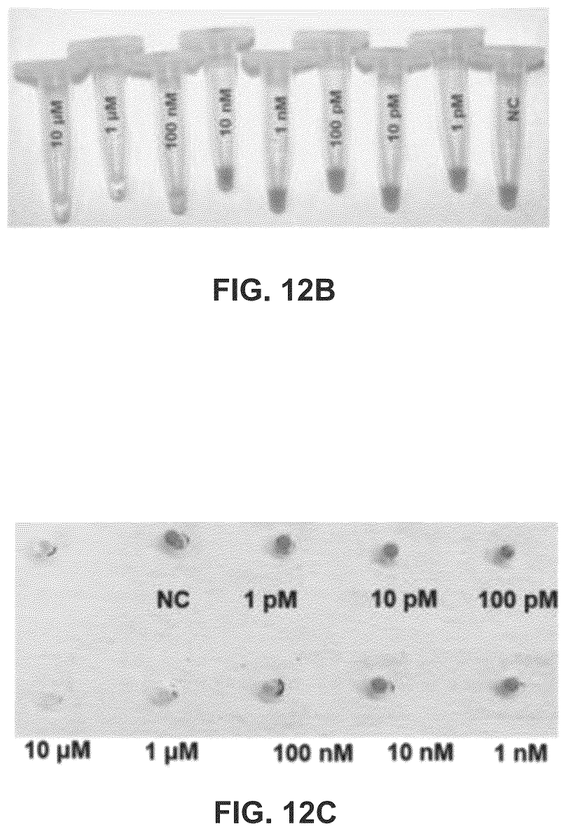

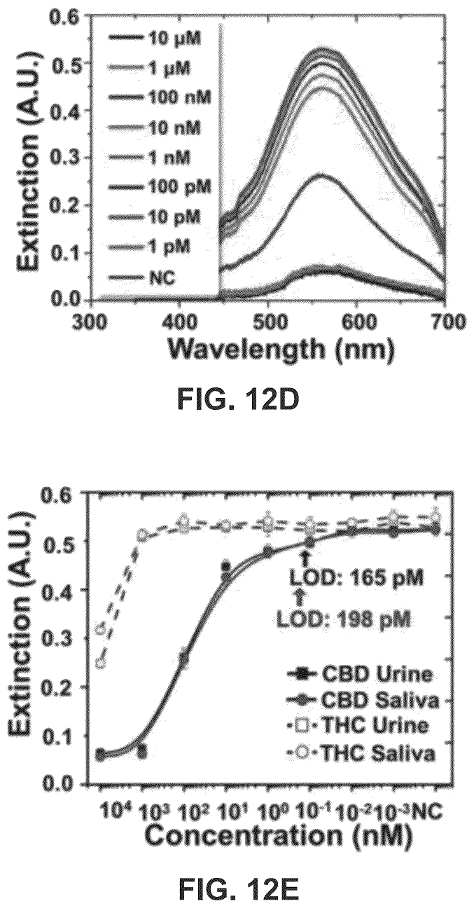

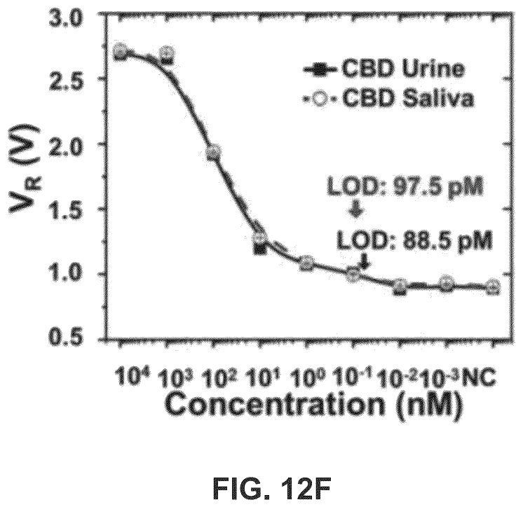

To further facilitate portable CBD detection, we demonstrated a homemade electronic readout system comprising a tandem LED and photodiode system (FIG. 12A). As described in our previous work, the LED emits at a wavelength matching that of the 80 nm AuNP extinction peak, and the light passing through the upper-level liquid is collected by a photodiode, where electrical signals is produced and adjusted using serially connected load resistor. For demonstration, a snug-fit microcentrifuge holder was 3d-printed, where windows were open to define the optical path along the upper portion of the liquid. The system was validated to produce a large dynamic range so that large variations in CBD concentration can be measured without saturation. In practice, we measured CBD in 5% urine and saliva, in both testing tubes (FIG. 12B) and PDMS well plate (FIG. 12C). The optical measurement results showed a LOD of ˜165 pM and ˜198 pM in urine and saliva (FIGS. 12D-12E), sample with a broad dynamic range (5 logs) and excellent specificity against THC. In comparison, the electronic measurement further improved the LOD to ˜88.5 and ˜97.5 pM, for urine and saliva, respectively, or ˜8 times better than tested by ELISA (FIG. 12F). This enhanced performance by electronic reading can be attributed to lower 3-σ errors generated during electronic measurements. With a small footprint, low-cost readout device (estimated <$5) and low reagent cost (estimated <$0.1 each test), reliable yet simple operation, and a potential for automatic data collection, our small molecule detection system has great potential for affordable mass applications.

### CONCLUSIONS

We have demonstrated a universal rapid assay design that combines synthetic, high-affinity, co-binding nanobodies and plasmonic nanoparticle transducers utilizing a portable electronic reader. Our approach has proven to be effective in detecting CBD, a very small molecule (Mw 314.47 g/mol) and an important molecular target in detection of drug misuse. Here we introduce a detection method that produces AuNP aggregation and sedimentation at presence of targeted small molecules, enabling CBD-concentration-dependent optical extinction display and subsequent electronic readout using simple electronic circuitry. Importantly, the IC-detect method (i.e. brief incubation followed by centrifugation prior to detection), was able to greatly reduce the detection time (less than 1.5 hours) by transporting small-molecules to the reaction zones at the testing tube bottoms, thus greatly decreasing both aggregation and sedimentation time. Additionally, this sensing system takes advantage of the multivalency of AuNP sensors and effective AuNP precipitation to overcome the traditional challenges of forming reliable readout signals with low-affinity small-molecule binders, achieving high sensitivity (<100 pM), large dynamic range (5 logs), and high specificity against THC in biological medium. Additionally, this assay format eliminates long incubation or cumbersome washing step typically required for ELISA or surface-based detection, and greatly decreased the footprint for readout, achieving highly sensitive, affordable and accessible detection in resource limited regions. Notably, the phage display selection method utilizes a premade combinatorial library that is applicable to a wide range of small molecules beyond CBD demonstrated in this work, making our sensing strategy applicable in diagnostics, therapeutic drug development, etc.

### Experimental Section

### Materials.

Phosphate-buffered saline (PBS) was purchased from Fisher Scientific. Bovine serum albumin (BSA) and molecular biology grade glycerol were purchased from Sigma-Aldrich. Sylgard 184 silicone elastomer kit was purchased from Dow Chemical. DNase/RNase-free distilled water used in experiments was purchased from Fisher Scientific. The Streptavidin-functionalized AuNPs were purchased from Cytodignostics, dispersed in 20% v/v glycerol and 1 wt % BSA buffer.

### Nanobody Generation, Selection, Expression, and Biotinylation.

In brief, rationally designed CDR sequences of different ratios of amino acids were used to generate a library of nanobodies. Anchor binders were selected after six rounds of biopanning using Biotin and Biotinylated-CBD-bound Streptavidin magnetic beads. Dimerization binders were selected after four rounds of biopanning using CBD-free and bound CA14 (anchor binder selected in the previous step) as the negative and positive control. All nanobodies were C-terminal AviTagged before Biotinylation. Nanobodies bearing AviTag were biotinylated by BirA using a BirA-500 kit (Avidity).

Preparation of Nanobody surface-functionalized AuNP colloidal solution. AuNPs of different sizes can be used for experimentation and optimization, here the methodologies are provided using 80 nm AuNPs.

Streptavidin surface functioned 80 nm-gold nanoparticles at 0.13 nM concentration were mixed with Biotinylated Nanobodies (both capture and different dimerization binders) in excess and incubated for 2 hours to ensure complete Streptavidin-Biotin conjugation. Next, centrifuge purification (accuSpin Micro 17, Thermo Fisher) was applied at 10,000 rpm for 10 mins and the top supernatant was discarded. This procedure was repeated twice to ensure high-quality purification. The purified AuNP colloidal solution concertation was measured by Nanodrop 2000 (Thermo Fisher) and readjusted to 0.048 nM, which was chosen for optimal extinction-level during spectrometric measurement. The buffer used for mixing, dilution, purification, and subsequent readjustment contained 1×PBS with 20 v % Glycerol and 1 wt % BSA, prepared from 10×PBS powder, Ultrapure water, Glycerol, and BSA. This buffer was used to ensure AuNP sensor stability and minimize nonspecific interactions.

Preparation of CBD and THC analyte solution. CBD and THC solutions were serially diluted from 10 UM to 1 pM in detection media, which typically contained 1×PBS with 20 v % Glycerol and 1 wt % BSA. For detection in urine and saliva, this buffer was readjusted to have 1×PBS with 20 v % Glycerol, 1 wt % BSA, and 20 v % of either urine or saliva.

Centrifuge enhanced rapid detection method. CBD solution with different concertation was pre-incubated for 2 hours with capture antibody functionalized AuNP (CA14-AuNP) colloidal solution in 2:3 volume ratio. This was done to make sure that we have maximum surface coverage with CBD-CA14-AuNP complex. Then chosen dimerization-binder-functionalized AuNP (DB-AuNP) were mixed with this pre-incubated colloidal solution at a composition volume ratio of CB:CA14-AuNP:DB-AuNP at 2:3:3. After mixing the solution was centrifuged at 3,500 rpm (1,200×g) for 1 minute. After a chosen incubation time (typically 20 mins), this colloidal solution was vortexed mildly at 800 rpm for 5 seconds.

PDMS well plate fabrication. PDMS well plate was created from Sylgard-184 elastomer. As detailed in our previous work, the PDMS was cured in a plain petri dish with a thickness of 2.5 mm. The PDMS film was cut into desired size and drilled 2 mm diameter holes with biopsy punch. Subsequently it was treated with oxygen plasma and bonded to a glass slide of desired size.

Spectrometric measurement. The UV-visible spectra and dark field imaging were performed using a customized optical system (Horiba), comprising an upright fluorescence microscope (Olympus BX53), a broadband 75 W Xenon lamp (PowerArc), an imaging spectrometer system (Horiba iHR320, spectral resolution 0.15 nm), a low-noise CCD spectrometer (Horiba Syncerity), a vision camera, a variety of filter cubes, operation software, and a high-power computer. Light transmitted through PDMS well plate was collected by a 50×objective lens (NA=0.8). The focal plane was chosen at the well plate surface to display the best contrast at the hole edge.

Electronic Measurement. An LED-photodiode electronic readout system was designed with three key components: an LED light source, a photodiode, and a microcentrifuge tube holder. The centrifuge tube holder was 3D printed using ABSplus P430 thermoplastic. An 8.6 mm diameter recess was designed to snuggly fit a standard 0.5 mL Eppendorf tube. 2.8 mm diameter holes were opened on two sides of the microcentrifuge tube holder to align a LED (597-3311-407NF, Dialight), the upper-level assay liquid, and a photodiode (SFH 2270R, Osram Opto Semiconductors). The LED was powered by two Duracell optimum AA batteries (3 V) through a serially connected 35 (2 resistor to set the LED operating point. The photodiode was reversely biased by three Duracell optimum AA batteries (4.5 V) and serially connected to a 7 MΩ load resistor. The photocurrent that responds to intensity of light transmitted through the assay was converted to voltage through the 7 MΩ load resistor and measured with a portable multimeter (AstroAI AM33D).

TEM sample preparation and imaging. To image AuNP precipitates, the supernatant was removed from the tube and 2 to 3 μL of AuNP sample colloid containing AuNP precipitates were left in the tube. The tube was then vortexed thoroughly. 2 μL of samples were pipetted and coated on both sides of the oxygen plasma-treated Cu grid (Electron Microscopy Sciences, C flat, hole size 1.2 μm, hole spacing 1.3 μm). The oxygen plasma treatment time was 30 seconds.

Some further aspects are defined in the following clauses:

Clause 1: A method of detecting cannabinoid molecules in a sample, the method comprising: contacting the sample with a plurality of plasmonic metal nanoparticles (MNPs) that are conjugated with at least two sets of antibodies, or antigen binding portions thereof, wherein at least a first set of antibodies, or antigen binding portions thereof, binds to a first epitope of a cannabinoid molecule and wherein at least a second set of antibodies, or antigen binding portions thereof, binds to a second epitope of the cannabinoid molecule under conditions sufficient for the first and second set of antibodies, or the antigen binding portions thereof, to bind to the first and second epitopes of the cannabinoid molecules in the sample to produce bound cannabinoid molecules; or contacting the sample with a plurality of plasmonic metal nanoparticles (MNPs) that are conjugated with a single set of antibodies, or antigen binding portions thereof, that bind to an identical epitope from different monomers of a dimerized cannabinoid molecule under conditions sufficient for the antibodies, or the antigen binding portions thereof, to bind to the identical epitope from the different monomers of the dimerized cannabinoid molecule in the sample to produce bound cannabinoid molecules; and, detecting the cannabinoid molecules when one or more aggregations of the bound cannabinoid molecules form with one another, thereby detecting the cannabinoid molecules in the sample.

Clause 2: The method of Clause 1, comprising contacting the sample with the plurality of MNPs conjugated with the first set of antibodies, or antigen binding portions thereof, under conditions sufficient for the first set of antibodies, or the antigen binding portions thereof, to bind to the first epitopes of the cannabinoid molecules in the sample to produce a first solution, and combining at least an aliquot of the first solution with the plurality of MNPs conjugated with the second set of antibodies, or antigen binding portions thereof, under conditions sufficient for the second set of antibodies, or the antigen binding portions thereof, to bind to the second epitopes of the cannabinoid molecules in the aliquot of the first solution.

Clause 3: The method of Clause 1 or Clause 2, wherein the antibodies comprise monoclonal antibodies.

Clause 4: The method of any one of the preceding Clauses 1-3, wherein the first and second set of antibodies, or the antigen binding portions thereof, comprise nanobodies.

Clause 5: The method of any one of the preceding Clauses 1-4, wherein the MNPs comprise gold nanoparticles (AuNPs).

Clause 6: The method of any one of the preceding Clauses 1-5, wherein the detection step comprises determining a change in absorbance at a resonance wavelength of the MNPs.

Clause 7: The method of any one of the preceding Clauses 1-6, comprising quantifying an amount of the cannabinoid molecules in the sample.

Clause 8: The method of any one of the preceding Clauses 1-7, further comprising centrifuging a solution comprising the sample and the plurality of MNPs to form MNP aggregations of the bound cannabinoid molecules prior to and/or during the detecting step.

Clause 9: The method of any one of the preceding Clauses 1-8, wherein the cannabinoid molecules are cannabidiol (CBD) molecules.

Clause 10: The method of any one of the preceding Clauses 1-9, wherein the cannabinoid molecules are tetrahydrocannabinol (THC) molecules.

Clause 11: The method of any one of the preceding Clauses 1-10, comprising obtaining the sample from a subject.

Clause 12: The method of any one of the preceding Clauses 1-11, comprising detecting the cannabinoid molecules within about 20 minutes or less of obtaining the sample from the subject.

Clause 13: The method of any one of the preceding Clauses 1-12, comprising repeating the method using one or more longitudinal samples obtained from the subject.

Clause 14: The method of any one of the preceding Clauses 1-13, wherein the sample comprises blood, plasma, serum, saliva, sputum, or urine.

Clause 15: The method of any one of the preceding Clauses 1-14, wherein the detecting step comprises measuring a colorimetric change when MNP aggregations of the bound cannabinoid molecules form with one another.

Clause 16: The method of any one of the preceding Clauses 1-15, comprising visually detecting the colorimetric change when the MNP aggregations of the bound cannabinoid molecules form with one another.

Clause 17: The method of any one of the preceding Clauses 1-16, comprising detecting the colorimetric change when the MNP aggregations of the bound cannabinoid molecules form with one another using a spectrometer.

Clause 18: A device, comprising at least one reaction chamber or substrate comprising a plurality of plasmonic metal nanoparticles (MNPs) that are (i) conjugated with at least two sets of antibodies, or antigen binding portions thereof, wherein at least a first set of antibodies, or antigen binding portions thereof, binds to a first epitope of a cannabinoid molecule and wherein at least a second set of antibodies, or antigen binding portions thereof, binds to a second epitope of the cannabinoid molecule when the reaction chamber or substrate receives a sample that comprises the cannabinoid molecules under conditions sufficient for the first and second set of antibodies, or the antigen binding portions thereof, to bind to the first and second epitopes of the cannabinoid molecule in the sample to produce bound cannabinoid molecules and one or more aggregations of the bound cannabinoid molecule to produce a colorimetric change in the reaction chamber; or (ii) conjugated with a single set of antibodies, or antigen binding portions thereof, that bind to an identical epitope from different monomers of a dimerized cannabinoid molecule under conditions sufficient for the antibodies, or the antigen binding portions thereof, to bind to the identical epitope from the different monomers of the dimerized cannabinoid molecule in the sample to produce bound cannabinoid molecules and one or more aggregations of the bound cannabinoid molecules to produce a colorimetric change in the reaction chamber.

Clause 19: A reaction mixture, comprising: a sample comprising cannabinoid molecules; and, a plurality of plasmonic metal nanoparticles (MNPs) that are (i) conjugated with at least two sets of antibodies, or antigen binding portions thereof, wherein at least a first set of antibodies, or antigen binding portions thereof, binds to a first epitope of a cannabinoid molecule and wherein at least a second set of antibodies, or antigen binding portions thereof, binds to a second epitope of the cannabinoid molecule in the sample; or (ii) conjugated with a single set of antibodies, or antigen binding portions thereof, that bind to an identical epitope from different monomers of a dimerized cannabinoid molecule in the sample.

Clause 20: A composition, comprising: a plurality of plasmonic metal nanoparticles (MNPs) that are (i) conjugated with at least two sets of antibodies, or antigen binding portions thereof, wherein at least a first set of antibodies, or antigen binding portions thereof, binds to a first epitope of a cannabinoid molecule and wherein at least a second set of antibodies, or antigen binding portions thereof, binds to a second epitope of the cannabinoid molecule in the sample; or (ii) conjugated with a single set of antibodies, or antigen binding portions thereof, that bind to an identical epitope from different monomers of a dimerized cannabinoid molecule in the sample.

Clause 21: A system, comprising: a device, comprising at least one reaction chamber or substrate comprising a plurality of plasmonic metal nanoparticles (MNPs) that are (i) conjugated with at least two sets of antibodies, or antigen binding portions thereof, wherein at least a first set of antibodies, or antigen binding portions thereof, binds to a first epitope of a cannabinoid molecule and wherein at least a second set of antibodies, or antigen binding portions thereof, binds to a second epitope of the cannabinoid molecule when the reaction chamber receives a sample that comprises the cannabinoid molecule under conditions sufficient for the first and second set of antibodies, or the antigen binding portions thereof, to bind to the first and second epitopes of the cannabinoid molecules in the sample to produce bound cannabinoid molecules; or (ii) conjugated with a single set of antibodies, or antigen binding portions thereof, that bind to an identical epitope from different monomers of a dimerized cannabinoid molecule when the reaction chamber receives a sample that comprises the cannabinoid molecules under conditions sufficient for the antibodies, or the antigen binding portions thereof, to bind to the identical epitope from the different monomers of the dimerized cannabinoid molecule in the sample to produce bound cannabinoid molecules; and, an electromagnetic radiation detection apparatus positioned, or positionable, within sufficient proximity to the device to detect one or more colorimetric changes produced in or on the reaction chamber or substrate when one or more aggregations of the bound cannabinoid molecules form with one another in or on the reaction chamber or substrate.

Clause 22: The system of Clause 21, wherein the electromagnetic radiation detection apparatus comprises a spectrometer; wherein the electromagnetic radiation detection apparatus comprises a microscope; and/or wherein the electromagnetic radiation detection apparatus comprises a light-emitting diode (LED) that transmits light into and/or through the reaction chamber or substrate and a photodetector that detects light from the reaction chamber or substrate.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, kits, reaction mixtures, devices, and/or systems or other aspects thereof can be used in various combinations. All patents, patent applications, websites, other publications or documents, and the like cited herein are incorporated by reference in their entirety for all purposes to the same extent as if each individual item were specifically and individually indicated to be so incorporated by reference.

What is claimed is:

1. A method of detecting cannabinoid molecules in a sample, the method comprising:
contacting the sample with a plurality of plasmonic metal nanoparticles (MNPs) that are conjugated with at least two sets of antibodies, or antigen binding portions thereof, wherein at least a first set of antibodies, or antigen binding portions thereof, binds to a first epitope of a cannabinoid molecule and wherein at least a second set of antibodies, or antigen binding portions thereof, binds to a second epitope of the cannabinoid molecule under conditions sufficient for the first and second set of antibodies, or the antigen binding portions thereof, to bind to the first and second epitopes of the cannabinoid molecules in the sample to produce bound cannabinoid molecules; or
contacting the sample with a plurality of plasmonic metal nanoparticles (MNPs) that are conjugated with a single set of antibodies, or antigen binding portions thereof, that bind to an identical epitope from different monomers of a dimerized cannabinoid molecule under conditions sufficient for the antibodies, or the antigen binding portions thereof, to bind to the identical epitope from the different monomers of the dimerized cannabinoid molecule in the sample to produce bound cannabinoid molecules; and,
detecting the cannabinoid molecules when one or more aggregations of the bound cannabinoid molecules form with one another, thereby detecting the cannabinoid molecules in the sample.

2. The method of claim 1, comprising contacting the sample with the plurality of MNPs conjugated with the first set of antibodies, or antigen binding portions thereof, under conditions sufficient for the first set of antibodies, or the antigen binding portions thereof, to bind to the first epitopes of the cannabinoid molecules in the sample to produce a first solution, and combining at least an aliquot of the first solution with the plurality of MNPs conjugated with the second set of antibodies, or antigen binding portions thereof, under conditions sufficient for the second set of antibodies, or the antigen binding portions thereof, to bind to the second epitopes of the cannabinoid molecules in the aliquot of the first solution.

3. The method of claim 1, wherein the antibodies comprise monoclonal antibodies.

4. The method of claim 1, wherein the first and second set of antibodies, or the antigen binding portions thereof, comprise nanobodies.

5. The method of claim 1, wherein the MNPs comprise gold nanoparticles (AuNPs).

6. The method of claim 1, wherein the detection step comprises determining a change in absorbance at a resonance wavelength of the MNPs.

7. The method of claim 1, comprising quantifying an amount of the cannabinoid molecules in the sample.

8. The method of claim 1, further comprising centrifuging a solution comprising the sample and the plurality of MNPs to form MNP aggregations of the bound cannabinoid molecules prior to and/or during the detecting step.

9. The method of claim 1, wherein the cannabinoid molecules are cannabidiol (CBD) molecules.

10. The method of claim 1, wherein the cannabinoid molecules are tetrahydrocannabinol (THC) molecules.

11. The method of claim 1, comprising obtaining the sample from a subject.

12. The method of claim 11, comprising detecting the cannabinoid molecules within about 20 minutes or less of obtaining the sample from the subject.

13. The method of claim 11, comprising repeating the method using one or more longitudinal samples obtained from the subject.

14. The method of claim 11, wherein the sample comprises blood, plasma, serum, saliva, sputum, or urine.

15. The method of claim 1, wherein the detecting step comprises measuring a colorimetric change when MNP aggregations of the bound cannabinoid molecules form with one another.

16. The method of claim 15, comprising visually detecting the colorimetric change when the MNP aggregations of the bound cannabinoid molecules form with one another.

17. The method of claim 15, comprising detecting the colorimetric change when the MNP aggregations of the bound cannabinoid molecules form with one another using a spectrometer.

18. A device, comprising at least one reaction chamber or substrate comprising a plurality of plasmonic metal nanoparticles (MNPs) that are (i) conjugated with at least two sets of antibodies, or antigen binding portions thereof, wherein at least a first set of antibodies, or antigen binding portions thereof, binds to a first epitope of a cannabinoid molecule and wherein at least a second set of antibodies, or antigen binding portions thereof, binds to a second epitope of the cannabinoid molecule when the reaction chamber or substrate receives a sample that comprises the cannabinoid molecules under conditions sufficient for the first and second set of antibodies, or the antigen binding portions thereof, to bind to the first and second epitopes of the cannabinoid molecule in the sample to produce bound cannabinoid molecules and one or more aggregations of the bound cannabinoid molecule to produce a colorimetric change in the reaction chamber; or (ii) conjugated with a single set of antibodies, or antigen binding portions thereof, that bind to an identical epitope from different monomers of a dimerized cannabinoid molecule under conditions sufficient for the antibodies, or the antigen binding portions thereof, to bind to the identical epitope from the different monomers of the dimerized cannabinoid molecule in the sample to produce bound cannabinoid molecules and one or more aggregations of the bound cannabinoid molecules to produce a colorimetric change in the reaction chamber.

19. A system, comprising:
a device, comprising at least one reaction chamber or substrate comprising a plurality of plasmonic metal nanoparticles (MNPs) that are (i) conjugated with at least two sets of antibodies, or antigen binding portions thereof, wherein at least a first set of antibodies, or antigen binding portions thereof, binds to a first epitope of a cannabinoid molecule and wherein at least a second set of antibodies, or antigen binding portions thereof, binds to a second epitope of the cannabinoid molecule when the reaction chamber receives a sample that comprises the cannabinoid molecule under conditions sufficient for the first and second set of antibodies, or the antigen binding portions thereof, to bind to the first and second epitopes of the cannabinoid molecules in the sample to produce bound cannabinoid molecules; or (ii) conjugated with a single set of antibodies, or antigen binding portions thereof, that bind to an identical epitope from different monomers of a dimerized cannabinoid molecule when the reaction chamber receives a sample that comprises the cannabinoid molecules under conditions sufficient for the antibodies, or the antigen binding portions thereof, to bind to the identical epitope from the different monomers of the dimerized cannabinoid molecule in the sample to produce bound cannabinoid molecules; and,
an electromagnetic radiation detection apparatus positioned, or positionable, within sufficient proximity to the device to detect one or more colorimetric changes produced in or on the reaction chamber or substrate when one or more aggregations of the bound cannabinoid molecules form with one another in or on the reaction chamber or substrate.

20. The system of claim 19, wherein the electromagnetic radiation detection apparatus comprises a spectrometer; wherein the electromagnetic radiation detection apparatus comprises a microscope; and/or wherein the electromagnetic radiation detection apparatus comprises a light-emitting diode (LED) that transmits light into and/or through the reaction chamber or substrate and a photodetector that detects light from the reaction chamber or substrate.
